(19) United States
(12) Patent Application Publication
(10) Pub. No.: US 2024/0240132 A1
(43) Pub. Date: Jul. 18, 2024

(54) INCUBATOR FOR CELL CULTURES

(71) Applicant: EPPENDORF SE, Hamburg (DE)

(72) Inventors: Benjamin Paulsen, Lübeck (DE); Christoph Jolie, Hamburg (DE); Gregor Bechmann, Hamburg (DE)

(21) Appl. No.: 18/556,832

(22) PCT Filed: Apr. 27, 2022
(86) PCT No.: PCT/EP2022/061225

§ 371 Date: Oct. 23, 2023

(30) Foreign Application Priority Data
Apr. 27, 2021 (EP) 21170740.1

(51) Int. Cl. C12M 1/36; C12M 1/00; C12M 1/26; C12M 1/34

(52) U.S. Cl. CPC C12M 41/48; C12M 29/04; C12M 29/08; C12M 33/04; C12M 41/14; C12M 41/34

(57) ABSTRACT

The invention relates to an incubator comprising sensor means for detecting VOC contamination of the gas atmosphere of the interior of the incubator chamber.

The invention relates to an incubator for the growth of biological cells. The invention also relates to a system and method for measuring an incubator atmosphere.

Such incubators are used in biological and medical laboratories to maintain cells in cell culture under controlled environmental conditions, thus enabling the growth of living cells in vitro. For this purpose, the temperature and gas composition or humidity of the atmosphere inside an incubator chamber isolated from the environment are maintained at the desired values by the incubator's apparatus. Eukaryotic cells require CO₂ incubators. The atmosphere is formed by air with a certain CO₂ and O₂ content and humidity, and a suitable temperature is often 37° C.

An incubator is not only an ideal growth environment for cell cultures, but also for biological contaminants. The consequences of biological contamination of cell cultures cause far-reaching problems in biological research, vaccine production, personalized medicine and regenerative medicine applications. Consequences of biological contamination are thus loss of time and money, inaccurate or erroneous experimental results. There are also cases, for example in forensics or reproductive medicine, where the value of a single sample, especially cell(s) located in a cell culture vessel, is much higher than, for example, the value of the entire incubator, so that loss of the sample due to contamination must be avoided at all costs.

However, contamination of the incubator chamber can also have non-biological causes, and can in particular be contamination carried into the incubator chamber from the environment of the incubator. Non-biological contamination is possible, for example, as a con-sequence of the manufacture of the incubator, if, for example, organic substances evaporate or are released from plastics, in particular from insulating and/or sealing material, and enter the incubator chamber.

The functional scope of prior art incubators often includes heating the incubator chamber, e.g. to 180° C., for the purpose of high temperature sterilization. For high-temperature sterilization, the incubator chamber must not contain any heat-sensitive objects. Therefore, the incubator chamber must be cleared out prior to high-temperature sterilization, and the sensitive specimens must be transferred to at least a second incubator during this process. This additional manipulation of the specimens creates an increased risk of specimen loss. In addition, there is a risk of introducing any contamination that may have arisen in the incubator into the at least one second incubator through the relocated sample containers, and possibly spreading it even further. In any case, a prudently performed high-temperature sterilization in combination with the sample rearrangement mean a considerable effort. Due to the importance of the sterility of the incubator chamber, it is nevertheless a standard recommendation of the manufacturers to perform the high-temperature sterilization at regular intervals, e.g. twice a month. In this way, statistically, the risk of sample loss due to contamination is reduced. It would be desirable to reduce the effort required by users to operate the incubator chamber as sterilely as possible.

The present invention is therefore based on the task of providing an incubator whose incubator chamber can be reliably operated by the user with as little effort as possible with low contamination.

The invention solves this problem by the incubator according to claim 1, the method according to claim 16 and a retrofittable sensor device according to claim 17. Preferred embodiments are in particular objects of the subclaims.

The incubator according to the invention for incubating living cell cultures, comprises: an incubator chamber for accommodating objects, in particular cell culture containers, in a closable interior space of the incubator chamber, which is operable with a controlled gas atmosphere, a sensor device for detecting an accumulation, in particular contamination, of volatile organic compounds (VOCs) in the gas atmosphere of the interior space, which sensor device comprises at least one VOC sensor for detecting the VOCs, which sensor comprises a measurement area which is arranged in flow communication with the atmospheric gas of the interior space.

The invention is based on the idea of performing a high-temperature sterilization of the incubator chamber not “blindly” periodically, but under automatically detectable conditions when a contamination occurs in the incubator chamber. For this purpose, the contamination should be detected, by means of contamination detectors (VOC sensors, see below), and preferably the user should also be informed immediately about a detected contamination, so that he can initiate the high-temperature sterilization. This results in the possibility of carrying out sterilization under objective conditions as required, and thus with the greatest efficiency.

It is known that certain contaminants are released into the environment as a result of contamination processes, in particular the growth of bacteria and microorganisms. The volatile organic gaseous metabolites of microorganisms are called MVOC (Microbial Volatile Organic Compounds) and can be used as indicators of microbial growth. In the context of the present invention, in a generalization of the concept of MVOC detection, all substances released into the environment or into the incubator atmosphere by chemical processes, in particular contamination processes, are referred to as VOCs (Volatile Organic Compounds).

From the prior art WO 97/08337 A a single vessel monitoring function in incubators is known, which have a passage opening of the incubator chamber, in order to take gas from this excess air area by means of a gas line, which is introduced into an excess air area of a vessel located above the sample volume in the incubator chamber, and to transport it to a VOC sensor device located outside the incubator. Since a vessel atmosphere has formed in the gas of the air supernatant area, in which VOC traces from the cell culture contained in the vessel may be enriched, this measuring arrangement is used to specifically detect contamination of the cell cultures contained in the vessel. However, it is not possible in this way to detect contamination that has formed in the incubator chamber itself, e.g. in a water tray of the incubator chamber, on the outside of door seals or incubated sample containers, or in other places that are susceptible to bacterial growth due to condensation-induced moisture formation.

In a research work underlying the present invention, a new arrangement has been developed in which the gas atmosphere of the interior of the incubator chamber, in which a plurality of sealed cell culture containers may be arranged, is tested by means of an incubator's own VOC sensor device, preferably as a regularly active monitoring function during the operating period of the incubator. Regardless of the origin of the contamination, users can be warned in this way and sterilization or cleaning of the interior of the incubator chamber can be initiated. In addition to contamination, an accumulation of VOCs in the incubator atmosphere can also be measured, in particular to detect certain biochemical processes.

The sensor device is used to detect contamination of the gas atmosphere of the interior. The sensor device preferably comprises at least one, preferably exactly one, VOC sensor for detecting at least one volatile organic compound (VOC). This VOC sensor is preferably designed or selected to be sensitive to at least one VOC released by a contamination and to measure whether the concentration of the VOC in the gas atmosphere of the interior space has changed, in particular increased.

The production of gaseous metabolic products by microorganisms and all other living organisms is a fundamental property. The volatile organic gaseous metabolites of microorganisms are referred to as MVOCs (Microbial Volatile Organic Compounds) and can be used as indicators of microbial growth, although their formation is mainly dependent on the species and the nutrient medium. Since cell cultures do not represent microbial organisms, the gaseous metabolites are referred to as VOCs. For simplicity, VOCs as well as MVOCs are referred to as VOCs in the context of this patent application. The term also includes—in particular—organic compounds that may evaporate from plastic parts e.g. in a period after manufacture. VOC does not refer to CO₂ gas (carbon dioxide gas), which is in particular a component of the gas atmosphere of the incubator chamber. Analogous to the VOCs of microorganisms, those of cell cultures can also be used as indicators of growth, as these provide information on the metabolic and physiological state of the cells.

The sensor device preferably comprises a plurality N with N>1 of VOC sensors, preferably a plurality N>=10 of VOC sensors. Preferably, the number of VOC sensors of the sensor device is limited to a maximum number M=2000, 100, 50, 25 or 10. The VOC sensors can all be of different types, resulting in a spectrum of type-different measurement results and thus a better differentiability of the VOC detection or a possible VOC detection or a possible VOC classification. Multiple VOC sensors of the same type may also be provided. For example, it is known from biology that the human nose uses about 380 different receptor types, while other mammals use a much higher number, so this approach can be considered technically “proven”.

Preferably, a sensor device is set up as an electronic nose, in which a plurality or multiplicity of VOC sensors measure, in particular simultaneously, the gas atmosphere of the interior, in particular by simultaneously detecting the accumulation of VOCs from the gas atmosphere at the measuring ranges of the VOC sensors. In particular, an electronic nose is suitable or arranged for collected measurement signals of the multiple or plurality N of VOC sensors, in particular each measurement signal of each of the VOC sensors, to be evaluated together in order to detect a contamination, in particular to detect the type or a class of a contamination, preferably also to quantify it. An electronic nose is particularly adapted to distinguish at least two VOCs present in the gas atmosphere. An electronic nose is particularly adapted to differentiate between different types or classes of contamination. Furthermore, a more reliable and thus also more sensitive detection of contamination can be achieved by means of multiple VOC sensors than with a single VOC sensor.

Preferably, the sensor device comprises at least one VOC sensor that is set up for the detection of gaseous metabolic products of microorganisms, in particular bacteria, mycoplasmas, fungi, yeasts. Preferably, the sensor device comprises several VOC sensors, in particular of different selectivity of detection.

Preferably, the sensor device comprises at least one VOC sensor that is set up for the detection of gaseous metabolic products of biological cells. This makes it possible to obtain information about the growth status of a cell culture, in particular its development over time.

Preferably, the sensor device comprises at least one VOC sensor that is set up for detecting gaseous substances that escape from the material of incubator components, in particular in a certain period after the manufacture of the incubator. These gaseous substances may include solvents or plasticizers, or other substances that escape from plastic components of the incubator.

Preferably, the sensor device comprises at least one VOC sensor that is set up to detect at least one chemical compound belonging to the alcohols, preferably several different chemical compounds belonging to the alcohols. In the research work on which this invention is based, it was found that alcohols can be detected particularly sensitively in the context of a contamination detection. This is due to the fact that metabolic processes of bacteria, which are particularly relevant for the contamination of incubators, produce alcohols to a special extent. On the other hand, the corresponding commercially available alcohol sensors are suitably sensitive to detect even small traces of alcohol.

Preferably, the sensor device comprises at least one VOC sensor that is set up to detect at least one chemical compound belonging to the aromatics, preferably several different chemical compounds belonging to the aromatics. In the research work on which this invention is based, it was also found in particular that aromatics can also be detected particularly sensitively in the context of a contamination detection. This is also due to the fact that metabolic processes of bacteria, which are particularly relevant for the contamination of incubators, produce various aromatics. On the other hand, the corresponding commercially available aromatic sensors are suitably sensitive to detect even small traces of aromatics.

Preferably, the sensor device comprises at least one VOC sensor that is set up to detect at least one chemical compound belonging to the alkanes, preferably several different chemical compounds belonging to the alkanes. In the research work on which this invention is based, it was also found in particular that alkanes can also be detected particularly sensitively in the context of a contamination detection. This is also due to the fact that metabolic processes of bacteria, which are particularly relevant for the contamination of incubators, generate various alkanes. This also applies in particular to metabolic processes of cells. On the other hand, the corresponding commercially available alkane sensors are suitably sensitive to detect even small traces of alkane.

Particularly preferably, the VOC sensor is set up to detect alcohols, and aromatics and/or alkanes.

Preferably, the sensor device comprises at least one VOC sensor which is set up for the detection of at least one of the chemical compounds, preferably several different chemical compounds belonging to the group of chemical compounds comprising {alcohols, aromatics, benzenes, alkylbenzenes, alkanes, alkenes, alkanes, aldehydes, esters, ketones, pyrazoles, oximes, terpenes, acids, carboxylic acids, heterocyclic amines and indoles}. In the research work on which this invention is based, it was also found in particular that these substances can be detected particularly sensitively in the context of a contamination detection. This is also due to the fact that metabolic processes of bacteria, which are particularly relevant for the contamination of incubators, generate various such substances. This also applies in particular to metabolic processes of cells. On the other hand, the corresponding commercially available sensors are suitably sensitive for various substances in order to detect even small traces of these substances.

A VOC sensor is in particular a chemical sensor for the detection of VOCs in a gas atmosphere. The similar or different VOCs detected in the gas atmosphere are detected by the sensor and converted into an electrical signal.

A VOC sensor can be a conductivity sensor, which in particular measures an electrical conductivity that varies as a function of at least one VOC. The VOC sensor is particularly preferably a metal oxide semiconductor (MOX) gas sensor, also referred to as a MOX sensor for short. Such chemical sensors comprise a detection layer by means of which a chemical interaction can be transformed into an electrical signal. They are suitable for continuous measurement operation.

The function of a MOX sensor is based in particular on the fact that, depending on the concentration of the target gas, the electrical conductivity of the gas-sensitive metal oxide layer or semiconductor changes and thus the presence as well as the quantity of the target gas is determined. Typically, a MOX sensor consists of four elements: Gas sensitive metal oxide layer, electrodes, heating element and insulation layer. The heating element is separated from the gas-sensitive metal oxide layer and the contact electrodes by the insulation layer. The gas-sensitive metal oxide layer is heated by the heating element and oxygen molecules from the environment are adsorbed on the surface of the gas-sensitive metal oxide layer. The adsorbed oxygen molecules capture electrons from the conductive bands of the semiconductor and energetic barriers are formed, thus blocking part of the electron flow in the semiconductor and thus degrading the electrical conductivity or increasing the resistance of the gas sensor. As soon as reducing gases (target gases) are present, they react with the bound oxygen molecules. The oxygen molecules are released from the surface of the gas-sensitive metal oxide layer and the conductivity increases or the resistance decreases.

A VOC sensor can be a capacitive sensor, which in particular measures an electrical capacitance that changes as a function of at least one VOC. A VOC sensor can be an optical sensor that in particular measures an optical property that changes as a function of at least one VOC, e.g., a changing refractive index, a changing light intensity, or a changing light spectrum, wherein the wavelength of the light used is not limited and in particular also includes infrared. A VOC sensor may be a mass-sensitive sensor that measures, in particular, a changing mass as a function of at least one VOC, e.g., by detecting a changing vibration of a vibrating body interacting with the at least one VOC.

The sensor device is preferably a component of the incubator, and is in particular arranged in a housing or housing part of the incubator.

Preferably, an electronic evaluation device is provided, which preferably includes a data processing device. The evaluation device is set up to record at least one measurement signal, in particular measurement value, of the at least one VOC sensor and in particular to evaluate it. The evaluation device is preferably a component of the incubator, and is in particular arranged in a housing or housing part of the incubator. The evaluation device can also be a component of the sensor device, in particular if the latter is set up as part of a retrofit system for an incubator which, in particular, does not yet comprise such a sensor device.

The evaluation device, in particular the incubator, preferably comprises a data storage device connected to the data processing device for the exchange of data.

The evaluation device, in particular the data processing device, is preferably programmable with a program code, and in particular programmed to perform the following steps, in particular according to this program code:
Receiving at least one measurement signal, in particular measurement data, of at least one VOC sensor; in particular: Receiving a sequence of measurement signals in time, in particular for the duration of a measurement time Δτ, one after the other, which in particular form the time course of the measurement of at least one VOC sensor;
Comparing of the at least one measurement signal with at least one reference value;
Deciding, based on the result of this comparison, whether there is a change in the VOC concentration in the gas atmosphere of the interior, especially a change characteristic of contamination of the interior.

Said at least one reference value may be predetermined and may be stored in the data storage device. Said at least one reference value may also be a start value resulting from a measurement at the beginning of the measurement time Δτ during which the at least one VOC sensor detects the VOC molecules in the gas atmosphere of the interior. The start value of a VOC sensor can be detected in particular when the measuring range of the respective VOC sensor has been initialized, in particular by purging the measuring range with a purge gas, e.g. nitrogen N2. This purging is preferably carried out until the temporal course of the measurement signal of the at least one VOC sensor is constant or has a known reference course, e.g. is linearly increasing or decreasing.

The evaluation device, in particular the data processing device, is preferably programmed to perform the following steps, in particular according to this program code:
Receiving a measurement signal, in particular a measurement signal representing a resistance value, in particular an electrical resistance or an electrical conductivity of the measurement range, in particular measurement data, of the MOX sensor; in particular: Receiving a sequence of measurement signals in time, in particular for the duration of a measurement time Δτ, one after the other, which in particular form the time course of the measurement of the one MOX sensor;
preferably: Evaluating the measurement signal or signals to obtain one or more measurement signals therefrom again;
Comparing of the measurement signal or signals with at least one reference value;
Decide, based on the result of this comparison, whether there is a change in the VOC concentration in the gas atmosphere of the interior, especially a change characteristic of contamination of the interior.

A control device of the incubator, in particular a data processing device, is preferably set up and/or programmed to drive a VOC sensor—or several—with a constant voltage.

Particularly preferably, a control device of the incubator, in particular a data processing device, is set up and/or programmed to control a VOC sensor—or several—with periodically changing voltage. Since the yield of chemical reactions at a VOC sensor may depend on this voltage, or may even depend VOC-specifically, this periodic control is an advantageous operating mode of the sensor device.

A control device of the incubator, in particular a data processing device, is preferably set up and/or programmed to control a heating element of a MOX sensor with a periodically changing voltage in order to generate a corresponding periodically changing temperature at the metal oxide surface. This mode of operation of a sensor device is also referred to as “temperature cycled operation” (“TCO”). For this purpose, the heater is controlled here with a voltage U-H-Soll having a step-like progression, which provides several different values per heating period T. Each of these voltage values is preferably set to a different value. Each of these voltage values is preferably set for a predetermined portion of the heating period T. The heating period is preferably between 1 second [s] and 60 s, preferably between 5 s and 30 s, preferably between 15 s and 25 s, preferably between 17 s and 23 s, and is here 20 s. The control results in a periodically changing measurement signal with the measurement period T. The heating period can be selected in particular as a function of the thermal mass of the VOC sensor.

In the case of a periodic measuring signal, the evaluation is preferably performed by statistically evaluating one or preferably several periods of the measuring signal to obtain an (evaluated) measuring signal. Preferably, the data processing device is programmed to determine an average course of a measurement period. This may involve superposing the values of measurement signals of a number M of measurement periods and then multiplying this added period course by the inverse number 1/M. In this way, a measurement signal is smoothed and the influence of measurement artifacts is reduced. A median filter can also be used for smoothing. Smoothing can also be performed by combining several successive measurement signals, in particular of a measurement period, as a mean value, thus reducing the number of measurement signals (then mean values) of a measurement curve; this is also known as moving average.

Preferably, the data processing device is programmed to derive from the signal of a single measurement period or from an average course of a measurement period at least one secondary value relating to a characteristic of the measurement period referred to as a secondary feature. A secondary value may be a slope which is present at a characteristic time of the measurement period, for example the time of the changeover of the voltage value. In particular, the characteristic slope can be recorded shortly before or after these times, since the slope can be undefined during the changeover. For the purpose of further evaluation, the secondary value can be compared as the new measurement signal, as described, with at least one reference value for this secondary value.

Preferably, the data processing device is programmed to determine an average value of several measurement signals, in particular, to determine an average value of several or essentially all measurement signals of a measurement period. For the purpose of further evaluation, the mean value can be compared with at least one reference value for this mean value, as described.

The incubator, in particular the sensor device, preferably comprises an electronic control device, which in particular can comprise a second data processing device or which uses the data processing device of the sensor device and which is programmed to control at least one valve, by the opening of which a purge gas, in particular N₂, flows over the measuring range of the at least one VOC sensor. This valve can be opened and/or closed in particular as a function of the measurement signals of the at least one VOC sensor.

The evaluation device, in particular the data processing device, is preferably programmable, and in particular programmed to perform the following steps:
Receiving at least one measured value, in particular measured data, from at least two, more or all of a plurality or plurality of VOC sensors; in particular for each of said at least two VOC sensors;
in particular in parallel over time: receiving a sequence of measured values of at least one VOC sensor one after the other over time, in particular for the duration of a measuring time Δτ, which in particular form the time course of the measurement of at least one VOC sensor;
Comparing of the at least one measured value of each of the at least two VOC sensors with at least one reference value;
Deciding, based on the result of this comparison, whether there is a change in the VOC concentration in the gas atmosphere of the interior, especially a change characteristic of contamination of the interior.

The at least one reference value can be determined in particular by flushing the at least one measuring range of the at least one VOC sensor with a flushing gas, in particular nitrogen, in a period before or after the measurement of the gas volume of the incubator atmosphere, and performing the reference measurement to detect one or more reference values at the measuring range flushed with flushing gas. The comparison of the measured value and the reference value—with optional additional consideration of a routinely selectable tolerance value—reliably leads to the detection of VOCs in the gas atmosphere of an incubator chamber.

As was found out in the course of the research work on which this invention is based, bacterially induced accumulations of VOCs in particular, i.e. contaminations, can be easily detected in the gas atmosphere of an incubator chamber, since bacteria, due to their exponential growth in the corresponding log phase, exhibit an exponentially growing metabolism with a correspondingly exponentially growing release of VOCs. In comparison, the emission of VOCs from normally growing cell cultures is very low, insofar as, for example, a proportion of these “normal” VOCs enters the gas atmosphere of the incubator chamber through cell culture containers that are not tightly sealed, e.g. Petri dishes. The bacterial metabolism thus dominates the VOC-related “odor” of the gas atmosphere of an incubator chamber.

The evaluation device, in particular the data processing device, is preferably programmable, and in particular programmed to perform the following steps or at least one of the following steps:
optionally: generating result data containing information on whether there is a change in the VOC concentration in the gas atmosphere of the interior, in particular a change characteristic of contamination of the interior;
optionally: output of information to a user of the incubator as a function of the result data as to whether there is a change in the VOC concentration in the gas atmosphere of the interior, in particular a change which is characteristic of a contamination of the interior; the output of this information can be in the form of visual or acoustic signals which are generated by means of a suitable output device of the incubator, in particular a display, and/or a loudspeaker. This output can be designed in particular as a warning signal to warn the user of an existing contamination;
optional: save the result data in the data storage device;
optionally: transmitting the result data to an external data processing device, in particular to a PC, a smartphone, a tablet computer, in particular by means of a wired or wireless signal connection.

A VOC sensor is used for the detection of volatile organic compounds (VOCs), the VOC sensor comprising a measurement area arranged in flow communication with the atmospheric gas of the interior chamber. Preferably, the measurement area of at least one VOC sensor or more than one or all of the VOC sensors is disposed within the incubator chamber. Preferably, the measurement area of at least one VOC sensor or of several or all VOC sensors is arranged in at least one or more measuring chamber(s). Preferably, the interior atmosphere of the measurement chamber is in flow communication with the gas atmosphere of the interior of the incubator chamber. This flow connection can be formed by connecting the incubator chamber and the measurement chamber by at least one flow channel. However, the measuring chamber can also be arranged in the incubator chamber, or the interior of the measuring chamber can open directly into the interior of the incubator chamber without requiring a dedicated flow channel. The section of a flow channel through which the at least one measurement area of the at least one VOC sensor is arranged may also be considered a measuring chamber. A measuring chamber may comprise an inflow opening through which the gas to be measured enters the measuring chamber and, in particular, an outflow opening through which the gas to be measured exits the measuring chamber.

In particular, the sensor device comprises at least one temperature sensor and/or one humidity sensor and/or one pressure sensor, which is preferably arranged in the measuring chamber for measuring the gas atmosphere.

The at least one measurement area of the at least one VOC sensor is preferably arranged in a section of the measuring chamber which, viewed along a straight connection between an inflow opening and an outflow opening of the measuring chamber, lies between this inflow opening and this outflow opening. The at least one measuring section of the at least one VOC sensor is preferably arranged parallel to a flow direction, in particular the main flow direction in a measuring chamber. This flow direction in particular follows a line, in particular a straight line, which connects the inflow opening and the outflow opening of the measuring chamber. The at least one measurement area of the at least one VOC sensor is preferably arranged parallel to a side wall of the measuring chamber and/or forms a side wall of the measuring chamber. In this context, a side wall is in particular an inner wall of the measuring chamber along which the flow of the gas/atmosphere to be measured flows. These measures ensure in particular that the concentration of VOCs from the gas atmosphere is as constant as possible along the measuring range by replacing a gas atmosphere that may have been depleted by the separation of VOCs at the measuring range with original gas atmosphere.

It is possible and preferred that the measuring ranges of several VOC sensors are arranged parallel to each other and in particular parallel to a flow direction through the measuring chamber. It is possible and preferred that the measurement areas of several VOC sensors are arranged parallel to each other and/or in particular parallel to a flow direction through the measuring chamber. Measurement areas can thereby surround a flow section through the measuring chamber, can in particular be arranged concentrically around this flow section or the flow direction. Measurement areas are arranged in particular around the flow section or the flow direction in such a way that a gas volume section located in the measuring chamber and flowing through the measuring chamber is simultaneously in contact with several or all measurement areas of several VOC sensors. This ensures, in particular, that the same gas composition prevails at the measurement areas and, in particular, that an upstream deposition of VOCs at an upstream sensor does not cause the downstream sensor to measure a different gas composition.

A flow channel is formed in particular by at least one gas line. One end of this pipe opens in particular into the incubator chamber, and the other end opens in particular into the measuring chamber.

At least one valve device can be provided to open or close, in particular also to throttle, the passage of the at least one flow channel, controlled by an electronic control device of the incubator or the sensor device. The valve device may in particular comprise at least one directional control valve, in particular at least one 3/2-way valve. The incubator or the sensor device may in particular comprise a purge gas reservoir containing purge gas, in particular N2 or a noble gas, for purging the measurement chamber and which is connected to the at least one flow channel by means of the valve. The at least one directional control valve can be set up to open the passage through the valve between the incubator chamber and the measurement chamber in a first switching position and simultaneously close the passage through the valve between the purge gas reservoir and the measurement chamber, and to close the passage through the valve between the incubator chamber and the measurement chamber in a second switching position and simultaneously open the passage through the valve between the purge gas reservoir and the measurement chamber. Instead of a purge gas reservoir, a connection for supplying purge gas can also be provided, to which a purge gas reservoir can be connected or to which a stream of purge gas (e.g., delivery of—in particular filtered—ambient air) can be supplied.

Preferably, the incubator or the sensor device comprises a gas conveying device, in particular a fan or a pump, by means of which gas atmosphere is conveyed from the incubator chamber through the flow channel into the measuring chamber. The measuring chamber preferably comprises a flow channel, in particular at least one gas conduit, through which gas atmosphere is conveyed out of the measuring chamber. In particular, gas atmosphere is conveyed out of the measuring chamber into the environment of the incubator, in particular into a housing area of the incubator that is open to the environment.

However, it is also possible and preferred that a flow channel is provided which guides the gas exiting the measuring chamber back into the incubator chamber. This prevents loss of incubation gases, in particular CO₂. Preferably, the returned gas is guided through a filter device, which may in particular comprise a HEPA filter. In particular, the filter device is set up to filter out substances and particles that have been added to the gas by the measuring chamber.

Preferably, at least one flow channel, in particular a gas line, which connects the incubator chamber and the measuring chamber upstream or downstream of the measuring chamber, in particular also at least a section of at least one measuring chamber outer side, is thermally insulated with a thermal insulating device. The thermal insulation device may comprise a double wall, or a thermally insulating material layer, which in particular may comprise air pockets with an insulating effect.

Preferably, at least one flow channel, in particular a gas line connecting the incubator chamber and the measuring chamber upstream or downstream of the measuring chamber, is in contact with a temperature control means, in particular a heating device, to heat the flow channel, in particular to the temperature of the incubator chamber or higher. In particular, the at least one flow channel can be guided along a wall of the—already tempered—incubator chamber. Such measures are intended to prevent condensation effects from occurring or VOCs from being altered or precipitated due to temperature.

The gas atmosphere of the interior of the incubator comprises, in particular, a different composition than the atmosphere in the cell culture containers arranged in the interior. In particular, the atmosphere in the cell culture containers is in direct contact with liquid growth media (cell media). In particular, the gas entering the measurement chamber and the gas atmosphere of the interior space comprise the same composition.

The measurement area of the at least one VOC sensor is preferably arranged in flow communication with the atmospheric gas of the interior space by being arranged in the incubator chamber, in which case in particular no separate measuring chamber would be necessary, but may be provided. The at least one measurement area can in particular be directly adjacent to the interior space of the incubator chamber and be directly in contact with the gas atmosphere of the interior space, wherein in particular a waste heat-generating component of the at least one VOC sensor is arranged outside the incubator chamber, and in particular in an environment of the incubator chamber. This environment is in particular not in contact with the measurement area and is in particular cooled, preferably air-cooled.

It is in particular preferred that the at least one VOC sensor is arranged partially and in particular not completely in the incubator chamber and/or in the measuring chamber. It is in particular preferred that at least one section of the at least one VOC sensor opposite the measuring range of a sensor is/are arranged outside the incubator chamber and/or the measuring chamber. In particular, it is preferred that at least one heating means or all heating means of the at least one VOC sensor is/are arranged outside the incubator chamber and/or the measuring chamber. In this way, the electrical connection of the at least one VOC sensor to an electrical control device is possible and, in particular, simplified. In addition, the temperature control or cooling of the side of the at least one VOC sensor facing away from the measurement area, in particular of the heating means, is possible and in particular simplified.

By means of the gas line, atmospheric gas in particular can be transported directly from the incubator chamber into the outer chamber of the incubator, or is transported directly in this way. The sensor device preferably comprises one or preferably several measuring chambers in which at least one measurement area of at least one VOC sensor is arranged. The flow channel opening into the incubator chamber at one end may open into a measuring chamber at another end, or may be divided to open into several measuring chambers with several ends. In particular, a number N with 10>=N>=1 of VOC sensors can be provided per measuring chamber, into which in particular the gas duct opens. Exactly one measuring range of a VOC sensor or exactly two measuring ranges of VOC sensors can be arranged in one measuring chamber. Atmospheric gas from the incubator chamber can thus be transported in particular into at least one—preferably each—measuring chamber and is transported—in particular controlled by an electrical control device.

Preferably, a measurement chamber comprises an exhaust duct arranged to convey exhaust air from the measurement chamber to an exterior space of the incubator chamber or incubator.

Preferably, the sensor device comprises a plurality of VOC sensors whose measurement areas, in particular whose adsorption areas, are arranged in contact with an interior space of the measuring chamber.

Preferably, the sensor device comprises a plurality of VOC sensors whose measurement areas, in particular whose adsorption areas for adsorption of VOCs to the measurement area, are arranged in contact with an interior space of the measuring chamber, the VOC sensors comprising a heating side which is arranged outside the measuring chamber in each case.

Preferably, the measuring chamber comprises a torus-shaped interior, in particular in the form of a closed torus, in particular with at least one inflow opening and at least one outflow opening. The torus can lie parallel to a plane, the inflow opening can be arranged essentially on this side of the plane and in particular on this side of the torus, and the outflow opening can be arranged essentially on the other side of the plane and in particular on the other side of the torus. However, the inflow opening and the outflow opening may also lie in the plane or cross the plane.

In particular, the incubator or the sensor device may comprise a conveying means for conveying gas, by means of which an atmospheric gas located in the interior of the measuring chamber and previously removed from the incubator chamber can be circulated. In this case, the inflow opening and/or the outflow opening may comprise a closure—in particular one that can be controlled by an electrical control device—with which the corresponding opening can be selectively closed. By circulating the gas, a gas exchange is achieved at the measurement areas/adsorption surfaces, and a longer-term discharge of gas atmosphere to be measured from the incubator chamber can be avoided. This can avoid the consumption of gas, e.g. CO₂, and energy that might otherwise be required to re-establish the incubator atmosphere in the incubator chamber and/or to equalize the pressure.

Preferably, the incubator and/or its electronic control device is set up to control a corresponding inflowing volume flow of at least one incubator gas, in particular a fixed mixture of incubator gases, e.g. N₂ and CO₂, as a function of the volume flow flowing out of the incubator chamber through a flow channel in the direction of a measuring chamber. In this way, the incubator atmosphere is kept as unchanged as possible—even during a measurement by means of the sensor device. The temperature of the incubator atmosphere, which may possibly change or drop as a result of this gas discharge and supply, can also be kept constant or readjusted, in particular to a target temperature, e.g. 37° C., by tempering by means of the temperature control device of the incubator, in particular its temperature control device(s) and temperature sensor(s).

Preferably, the sensor device is configured as an electronic nose. For this purpose, it comprises in particular an electronic control device and a plurality of VOC sensors, preferably of different types, and in particular a flushing device by means of which at least one measuring chamber can be flushed by a flushing gas.

Preferably, the electronic control device of a sensor device or incubator configured as an electronic nose comprises a data processing device comprising at least one data memory programmed to perform at least one, more or each of the following steps. Analogously, the method according to the invention may comprise these steps:
i) storing a measurement data record in the data memory which contains the measurement values of the number N>1 of VOC sensors which are recorded, in particular as a function of time, in particular within the same at least one time period, a measurement value being characteristic of the detected measurement signal of the respective VOC sensor which was recorded in the presence of a volume of the gas atmosphere which originates from the incubator chamber and is present at the measurement range of the VOC sensor, in particular flowing past;
i) determining first result measurement data from a comparison of the measurement data set with a reference data set, in particular using a difference of the measurement data set and the reference data set which contains the reference measurement values, in particular recorded as a function of time, of the number N>1 of VOC sensors, a reference measured value being characteristic of the detected measurement signal of the respective VOC sensor, which was recorded in the presence of a purge gas originating from a purge device and being in contact with, in particular flowing past, the measurement range of the VOC sensor;
iii) optionally: recognizing a characteristic data pattern in the result measurement data set containing the result measurement data, wherein the characteristic data pattern represents a specific VOC detected in the atmospheric gas, in particular also its concentration or amount.

The characteristic data pattern in the result measurement data set containing the result measurement data can preferably be taken into account by the following evaluation: a contamination can be present in particular if the measured values of a certain subset of VOC sensors of the sensor device—optionally taking into account a tolerance value—deviate from their respective reference value, —optionally a relative degree of deviation can be taken into account-, but that the measured values of the other VOC sensors of the sensor device—optionally taking into account a tolerance value—do not deviate from their respective reference value. The characteristic data pattern can be determined beforehand by measurements on one or more test gases with defined VOC content.

Step iii) may preferably involve using a classification algorithm determined by machine learning, in particular an artificial neural network, to classify the characteristic data pattern.

Preferably, the control device comprises a data processing device comprising at least one data memory programmed to perform at least the first, or more, or all of the following steps. Analogously, the method according to the invention may comprise these steps:
i) storing a test measurement data set in a data memory which contains test measurement values of the number N>1 of VOC sensors, a test measurement value being characteristic of the detected measurement signal of the respective VOC sensor, which was recorded in the presence of a volume, in particular flowing past, of a preknown test gas with a VOC content, in particular preknown in terms of type and/or quantity, which is supplied to the measurement chamber and is adjacent to the measurement range of the VOC sensor;
i) determining second result measurement data from a comparison of the test measurement data set with a reference data set, in particular using a difference of the test measurement data set and the reference data set, which contains the reference measurement values of the number N>1 of VOC sensors, a reference measurement value being characteristic of the detected measurement signal of the respective VOC sensor, which was recorded in the presence of a purge gas originating from a purge device and lying, in particular flowing past, the measurement range of the VOC sensor;
iii) storing a second result measurement data set containing the second result measurement data and comprising a now known data pattern characteristic of the test gas.

By means of the above steps, in particular, a method can be realized by means of which the presence of certain VOCs in the gas atmosphere can be detected, in particular the concentration of at least one VOC in the gas atmosphere can be estimated.

Preferably, a step iv) is carried out in which the second result measurement data obtained in iii) is used as labeled data to train an adaptive classification algorithm by machine learning, in particular a neural network, which can subsequently be used to classify measured characteristic data patterns. By means of this step, also in particular a method can be realized by means of which the presence of certain VOCs in the gas atmosphere can be detected, in particular the concentration of at least one VOC in the gas atmosphere can be estimated.

Preferably, the incubator comprises an information output system, in particular a display, a loudspeaker or a data interface to an external data processing device, in order to output information about the detection of VOCs as a function of the detection detected by means of the sensor device, in particular in order to output warning information to a user or a monitoring system.

The invention also relates to a laboratory monitoring system for detecting the contamination of at least one incubator chamber, comprising
at least one incubator according to the invention;
at least one data processing device arranged externally to the at least one incubator, which is in particular in a data exchange connection with the at least one incubator, in particular via an intranet or the Internet;
wherein the data-processing device is programmed to acquire the measurement data about a possible contamination of the incubator chamber obtained from the at least one incubator and determined by means of the sensor device of the incubator by the detection and to store said measurement data in a data storage device, in particular in order to communicate said measurement data to a further device, in particular to a PC, a mobile radio device, a smartphone or a tablet computer.

The invention also relates to a method for detecting contamination in the incubator chamber of an incubator, in particular an incubator according to the invention, comprising the steps:
collecting of measurement data by means of a sensor device comprising at least one VOC sensor for the detection of volatile organic compounds (VOCs), the VOC sensor comprising a measurement area which is arranged in flow communication with the atmospheric gas of the interior;
determining possible contamination of the gas atmosphere of the interior by evaluating the measurement data.

The invention also relates to a retrofit system with a retrofittable sensor device for detecting possible contamination of the gas atmosphere of the interior of an incubator chamber, wherein the sensor device comprises at least one VOC sensor (11) for detecting volatile organic compounds (VOCs), wherein the VOC sensor comprises a measurement area which is arrangeable in flow communication with the atmospheric gas of the interior space, and wherein the sensor device preferably comprises a gas line arrangeable between the interior space and the measurement area, and preferably a pump for conveying a volume of the gas atmosphere of the interior space of the incubator chamber through the gas line to the measurement area. The invention also relates to such a retrofittable sensor device for an incubator. The retrofit system or the retrofit sensor device further comprises, in particular, an electronic evaluation device which preferably includes a data processing device. The evaluation device is preferably set up to record at least one measurement signal, in particular measurement value, of the at least one VOC sensor and in particular to evaluate it. Alternatively and/or additionally, the retrofit system comprises a program code, during the execution of which a data processing device, in particular of the incubator, acquires and in particular evaluates the in particular one measured value of the at least one VOC sensor. The possible steps of a program code have already been/are still described here.

The invention also relates to an incubator arrangement comprising an incubator with an incubator chamber and a retrofittable sensor device as described above, which is arranged on the incubator or in the incubator chamber of the incubator for detecting possible contamination of the gas atmosphere of the interior of an incubator chamber.

The incubator is a laboratory device or a laboratory incubator. In particular, an incubator refers to a laboratory device with an incubator chamber whose atmosphere can be controlled or is controlled by the incubator to a predetermined target temperature. In particular, it is a laboratory device that can be used to create and maintain controlled climatic conditions for various biological development and growth processes. Preferably, the incubator may be or include a shaker, i.e., an incubator comprising a movement device for moving objects disposed in the incubator chamber. The incubator may be a cell cultivation device, a microbial incubator (also without CO₂). In particular, the incubator serves to create and maintain a microclimate with controlled gas, and/or humidity, and/or temperature conditions in the incubator chamber, which treatment may be time-dependent. The laboratory incubator, in particular a treatment device of the laboratory incubator, may in particular comprise a timer, in particular a timer, a heating/cooling device and preferably a setting for the regulation of an exchange gas supplied to the incubator chamber, an adjustment device for the composition of the gas in the incubator chamber of the incubator, in particular for adjusting the CO₂ and/or the O₂ and/or the N₂ content of the gas and/or an adjustment device for adjusting the humidity in the incubator chamber of the incubator. The incubator, in particular a treatment device of the incubator, comprises in particular the incubator chamber, further preferably a control device with at least one control loop, to which the at least one heating/cooling device is assigned as an actuator and at least one temperature measuring device is assigned as a measuring element. By means of the control device, the temperature in the incubator can be controlled. Depending on the embodiment, the humidity can also be controlled via it. A tub filled with water in the incubator chamber can be heated or cooled in order to adjust the humidity via evaporation. Alternatively and/or additionally, a water evaporator can be provided as part of the incubator, by means of which the humidity in the atmosphere of the incubator chamber is adjusted. CO₂ incubators are used in particular for the cultivation of animal or human cells. Incubators may comprise turning devices for turning the at least one cell culture container and/or a shaking device for shaking or moving the at least one cell culture container.

A sensor arrangement of the incubator, which can in particular be assigned to a control device, comprises in particular at least one temperature sensor, preferably a plurality of temperature sensors. A temperature sensor can be, for example, a Pt 100 or Pt 1000 temperature sensor. A sensor device preferably comprises a sensor for determining a relative gas concentration, in particular for determining the content of CO₂ and/or O₂ and/or N₂. A sensor device preferably comprises a sensor for determining the relative humidity of the air.

An incubator preferably comprises one or a single incubator chamber. This can be divided into compartments. Compartments can be separated by—in particular perforated—bearing plates, whereby in particular a gas exchange between the compartments is enabled. A bearing plate, in particular its lower side, can be set up to hold the sensor device or a flow channel by means of which incubator atmosphere is fed into a measuring chamber and may in particular comprise a holder for these parts.

The incubator chamber comprises chamber walls or chamber inner walls and exactly one or at least one chamber opening via which the objects or cell culture containers can be placed inside the incubator chamber and removed. This chamber opening is closable by a closure element movably connected to the incubator chamber, in particular an incubator door movably mounted on the incubator chamber by means of a hinge, in particular one or more chamber doors. An incubator may comprise one or more inner doors, which can in particular be transparent, and can comprise an—in particular non-transparent—outer door, which in particular thermally insulates the incubator chamber and possibly at least one inner incubator door, which closes or opens the chamber opening, from the environment.

In the closed position of the chamber opening, the interior (synonymously: the inner space) of the incubator chamber is preferably isolated from the environment in such a way that a desired atmosphere controlled by the incubator can be set, in particular controlled, in the interior. The term “gas atmosphere of the interior of the incubator chamber” does not refer to the interior of substantially closed hollow objects arranged in the incubator chamber, and in particular does not refer to the container interior of a container arranged in the incubator chamber, the opening of which is closed, in particular covered, or not closed in a gas-tight or gas-tight manner. This container interior, e.g. of a cell culture container, typically comprises a liquid medium and an air supernatant gelled above this liquid. In the open position of the chamber opening, gas exchange between the environment of the incubator and the interior of the incubator chamber is possible via this opening. The chamber opening is typically located in a front wall surrounding the chamber opening.

The incubator chamber preferably comprises a plurality of walls or inner wall surfaces which can be connected to one another, in particular integrally and in particular without edges. The walls or inner wall surfaces are preferably substantially planar in shape, but may also all or in part comprise a curved shape. The incubator chamber is preferably cuboidal in shape, but may also be otherwise shaped, e.g. spherical, ellipsoidal, polyhedral. The walls or inner wall surfaces are preferably made of a low-corrosion material, in particular stainless steel, copper, brass, or a plastic, in particular a composite plastic. This facilitates cleaning/sterilization of the chamber interior. Independent of the chamber opening, which is used for loading/removing objects or cell culture containers, the incubator chamber may comprise at least one port for passing an appropriately dimensioned device or a gas line and/or a cable connection from the interior of the incubator chamber to the outside thereof or to the environment of the incubator. A port includes in particular an opening in a chamber wall of the incubator chamber for the passage of an appropriately dimensioned device or a gas line and/or a cable connection from the interior of the incubator chamber to the outside thereof or to the surroundings of the incubator.

A typical size of the interior of an incubator chamber is between 50 and 400 liters.

The incubator may comprise exactly one incubator chamber, but may also comprise several incubator chambers whose atmosphere (temperature, relative gas concentration, humidity) may be adjustable, in particular individually or collectively. An incubator may have several incubator chambers, each of which may comprise its own chamber opening and its own chamber door for closing the chamber opening, in particular its own port. Several sensor devices may be provided, in particular one per chamber.

The incubator may comprise a housing that partially or completely surrounds the incubator chamber. The housing may be substantially cuboidal in shape, and may in particular be designed such that the incubator is stackable. The sensor device and/or the at least one VOC sensor is preferably arranged in the housing, or a housing section, in particular immovably connected thereto. In this way, the sensor device forms an integral part of the incubator. However, it can also be arranged as a module, in particular as part of a retrofit system in the housing, and in particular be immovably connected to it.

A storage area of the incubator is realized in particular by a storage plate, in particular a shelf plate insert, which can be made in particular of stainless steel or copper or similar or has this material. A storage plate serves as a floor plate, in particular as an intermediate floor plate. The bearing plate can be removable from the incubator chamber (“bearing plate insert”) or can be permanently inserted with it. The incubator chamber may comprise holding sections or a holding frame for holding one or more bearing plate inserts or insertable instruments. A bearing plate can be set up on its underside to hold a sensor device or at least one gas line, in particular comprise a holder for this sensor device or gas line. Alternatively or additionally, at least one of the inner walls of the incubator chamber may be arranged for holding one or more bearing plate inserts or insertable instruments. For this purpose, a retaining structure integrated into the wall may be provided, in particular one or more protrusions, grooves or webs. A storage plate increases the available storage area in the incubator chamber.

A holding frame for the at least one bearing plate is also preferably made of a non-corrosive material, preferably stainless steel. The holding frame is preferably designed as a standing object by comprising at least one base section that rests on the bottom wall of the incubator chamber. However, it may also be supported on the side walls of the incubator chamber and/or suspended from the ceiling wall of the incubator chamber.

A bearing plate preferably—and in particular substantially completely—extends across a horizontal cross-section of the incubator chamber.

Preferably, the incubator comprises a treatment device for treating the at least one cell culture container. The term “treatment” means in particular that an object, in particular a cell culture or a cell culture container is moved, and/or transported and/or examined and/or changed, in particular physically, chemically, biochemically or in any other way.

A treatment device may be a movement device by means of which the cell medium in at least one cell culture container is kept in motion, preferably via a movement program controlled by the control program. A movement device may be a shaking or pivoting device. A movement device preferably comprises a support device, in particular a plate, on which one or more cell culture containers are placed and/or fixed. A movement device preferably comprises a drive device, in particular in the case of a shaking device for example an oscillator drive, by means of which the desired movement program is implemented. The design of the movement program may depend on the growth stage of the cells of a cell culture and may depend on the cell type, in particular a cell line. The design and/or control of the treatment, in particular the movement program, may depend on the cell monitoring data. The One treatment device may be a pivoting device by means of which at least one cell culture container is pivoted. The components of the pivoting device may correspond to those of the shaking device, but are set up for a pivoting movement.

A treatment device can also be a transport device by means of which at least one cell culture container can be transported in the incubator chamber. The transport device can be a lift device comprising a carrier device on which the at least one cell culture container can be placed. The lift device preferably comprises a movement mechanism and/or an electrically controllable drive mechanism for driving the movement mechanism. The transport device may further be a movable and electrically controllable gripping arm for gripping and holding at least one cell culture container. The transport device may include a conveyor for moving the at least one cell culture container placed thereon. The transport may move the at least one cell culture container in the incubator chamber, in particular to a processing position in a processing station in the incubator chamber, and away from said processing position. The control device may be arranged to control the transport device in dependence on result data obtained by an evaluation device from the measured values of the sensor device.

The sensor device, in particular a gas line, can also be attached or fastened to a transport device located in the incubator chamber. The sensor device can be attached or fastened to a positioning mechanism by means of which the sensor device can be moved and positioned in the incubator chamber. The positioning mechanism may include a movable robotic arm and is preferably electrically controllable, in particular by a control program of the control device. In this way, a VOC concentration can be measured successively at different locations in the incubator chamber using one or a few sensor devices successively. The positioning mechanism may be configured as a component that can be inserted into the incubator chamber. The power supply of this component may be provided via a cable connection to the incubator, preferably via a cable connected through a wall opening, e.g. a port, or via such a cable connection to an external power source. The control device may be arranged to control the positioning mechanism in dependence on result data obtained by an evaluation device from the measured values of the sensor device.

The term treatment device can also be understood to mean the temperature control device of the incubator chamber, which is used to control the atmosphere inside the incubator chamber to the desired value, in particular 37° C. The term tempering refers to raising and lowering the temperature of the atmosphere by heating and cooling. Preferably, the temperature inside is adjusted by changing the temperature of the walls of the incubator. Temperature sensors of the corresponding temperature control device are distributed in at least one position inside and/or outside the incubator chamber, in particular on a wall of the incubator chamber.

The incubator preferably comprises a user interface device via which the user can input data to the data processing device or the control device, and/or via which information can be output to the user. Preferably, the incubator and/or this user interface device is arranged for the user to be able to receive information dependent on the measured values of the sensor device, in particular the result data of an evaluation device. Preferably, the incubator or this user interface device is arranged for the user to be able to enter at least one operating parameter for operating the incubator or the sensor device at this user interface device and/or to receive corresponding information therefrom. In this way, a single user interface device may be used by the user to influence, or control, or obtain information from, the incubator and also the at least one sensor device. In particular, the sensor device may be arranged to display to the user, in response to a user query made by means of the user interface device of the incubator, measured values or result data, or statistical information derived from measured values, and/or a time history of measured values or result data.

An equipment-controlled treatment of the incubator is preferably a program-controlled treatment, i.e., a treatment controlled by a program. By a program-controlled treatment of a sample it is to be understood that the process of treatment is essentially carried out by executing a plurality or a plurality of program steps. Preferably, the program-controlled treatment is performed using at least one program parameter, in particular at least one program parameter selected by a user. A parameter selected by a user is also referred to as a user parameter. Preferably, the program-controlled treatment is performed by means of the digital data processing device, which is in particular part of the control device. The data processing device may comprise at least one processor, i.e. a CPU, and/or comprise at least one microprocessor. Preferably, the program-controlled treatment is controlled and/or carried out according to the instructions of a program, in particular a control program. In particular, in a program-controlled treatment, substantially no user action is required at least after the program parameters required by the user have been acquired. A device-controlled treatment of the incubator can be carried out in particular in dependence on measured values or result data of the at least one sensor device.

A program parameter is a variable which can be set in a predetermined manner within a program or subprogram, valid for at least one execution (call) of the program or subprogram. The program parameter is set, e.g. by the user, and controls the program or subprogram and causes a data output depending on this program parameter. In particular, the program parameter and/or the data output by the program influences and/or controls the control of the device, in particular the control of the treatment by means of the at least one treatment device.

A program is understood to mean in particular a computer program. A program is a sequence of instructions, in particular consisting of declarations and instructions, in order to be able to process and/or solve a specific functionality, task or problem on a digital data processing device. A program is usually present as software to be used with a data processing device. In particular, the program may be present as firmware, in particular in the case of the present invention as firmware of the control device of the incubator or the system. The program is usually present on a data carrier as an executable program file, often in so-called machine code, which is loaded into the main memory of the computer of the data processing device for execution. The program is processed as a sequence of machine, i.e. processor, instructions by the processor(s) of the computer and is thus executed. By ‘computer program’ is understood in particular also the source code of the program, from which the executable code can arise in the course of the control of the laboratory device.

A user interface device may be a component of an incubator, or a module. A user interface device preferably comprises in each case: a control device for the user interface device; a communication device for establishing a data connection with a laboratory device, in particular an incubator, via an interface device thereof; an input device for detecting user inputs from a user; an output device, in particular a display and/or a display, for outputting information to the user, in particular a touch-sensitive display. In this context, the control device of the user interface device is preferably set up to exchange data with the control device of the incubator via the data connection.

An object that can be stored in the incubator chamber is in particular a cell culture container. A cell culture container is in particular transparent. In particular, it is made of plastic, in particular PE or PS, and in particular comprises a planar base plate which forms the growth surface of the cells. This may have a surface treatment to promote cell adherence. The cell culture container can be closed or provided with a PE cap or gas exchange cap, in particular a lid with optionally included filter. In particular, the cell culture container is stackable. An Eppendorf cell culture bottle is particularly suitable.

The incubator preferably comprises an electrical control device (synonym: control device), which may in particular include a control device. A data processing device is preferably part of a control device of the incubator, which controls functions of the incubator. The functions of the control device are implemented in particular by electronic circuits. The control device may include a microprocessor, which may include the data processing device. The control device and/or the data processing device is preferably configured to perform a control procedure, also referred to as control software or control program. The functions of the incubator and/or the control device and/or the evaluation device may be described in method steps. They can be realized as components of a computer program (code) of the control program, in particular as subroutines of the control program.

In the context of the present invention, a control device generally comprises or is in particular the data processing device, in particular a computing unit (CPU) for processing data and/or a microprocessor. The data processing device of the control device of the incubator is preferably also arranged for controlling a treatment process and/or individual treatments carried out by one or more, in particular optional, treatment devices of the incubator.

The data processing device is alternatively preferably a device located outside of and separate from the incubator, also referred to as an external device or external data processing device. The data processing device and the incubator are preferably in a data connection and are preferably components of a network for data exchange. In this case, the data processing device and the incubator are in particular components of a laboratory monitoring system according to the invention for detecting the accumulation of VOCs, in particular for detecting contamination.

In particular, the incubator comprises a control device with at least one control loop, to which the at least one temperature control device is assigned as an actuator and at least one temperature sensor is assigned as a measuring element. Depending on the embodiment, the air humidity can also be controlled via this, although the air humidity itself is not measured by an air humidity sensor (rH sensor) and the air humidity is not an input variable of the control loop. A water-filled tray in the incubator chamber can be heated or cooled to adjust humidity via evaporation. CO₂ incubators are used in particular for the cultivation of animal or human cells. Incubators can comprise turning devices for turning the at least one cell culture container and/or a shaking device for shaking or moving the at least one cell culture container.

When arranging a sensor device on or in the incubator chamber, it should be noted that the operation of electrical devices within the incubator chamber leads to waste heat which can heat the chamber atmosphere to an unacceptable extent. The control device of the incubator can therefore be set up in particular to control the operation of the electrical devices, in particular of the at least one sensor device inside or on the incubator chamber, as a function of temperatures of the chamber atmosphere detected by means of temperature sensors. The control device of the incubator can in particular be set up to control the temperature control of the chamber atmosphere by means of the at least one temperature control device and the operation of the electrical devices inside the incubator chamber as a function of one another in order to compensate for the undesired heating of the chamber atmosphere.

The control device may be arranged for a program parameter or a control parameter of the incubator to be selected automatically in dependence on other data. In an incubator, a treatment of the at least one cell culture in at least one cell culture container controlled by a control parameter corresponds in particular to a climate treatment to which the at least one cell culture is subjected. Possible parameters, in particular program parameters, in particular user parameters, used to influence a climatic treatment define in particular the temperature of the incubator chamber in which the at least one sample is incubated, the relative gas concentration of O₂- and/or CO₂ and/or N₂ in the incubation chamber, the humidity in the incubation chamber and/or at least one sequence parameter which influences or defines the sequence, in particular the order, of an incubation treatment program consisting of several steps.

The temperature control unit can be a combined heating/cooling unit. It is preferably only a heating device. In particular, this can generate the heat via an electrical resistance wire.

The processes controlled by the incubator also include all tempering steps that influence the physical state “temperature of the incubator atmosphere”, in particular also steps of an optional automatic sterilization by means of high-temperature phases at approx. 100° C. to 120° C. or up to 180° C. or 200° C., which are carried out with the incubator chamber empty.

The processes controlled by the incubator can further include gas exchange processes in which parts of the incubator atmosphere are exchanged according to a volume flow with a predetermined volume per time in order to set the desired gas composition of the incubator atmosphere, in particular as part of a control process, especially if this has changed in an undesirable manner after opening the incubator door or if an extraction of gas atmosphere of the incubator chamber in a measuring chamber changes the chamber atmosphere, in particular the chamber pressure, in an undesirable manner. The corresponding physical condition to be measured in this case is the relative gas concentration or relative humidity. The incubator or its control device is preferably set up to regulate the relative gas concentration or the gas value detected as a result of the relative gas concentration, in particular CO₂ and/or O₂ values, which is applied as a setpoint value of a temperature control loop at a specific time. This gas control can also include the control of the air humidity, which is carried out by means of an air humidity sensor of a sensor arrangement, in particular by means of a sensor for measuring the relative air humidity.

The processes controlled by the incubator itself may further include gas movement processes in which, for example, parts of the incubator atmosphere are moved according to a volumetric flow with a predetermined volume per time and in particular also in one or more variable or constant flow directions in the incubator chamber. This can lead in particular to a more uniform atmosphere within the incubator chamber, in particular in order to expose differently positioned cell culture containers to the same atmosphere on a time-average basis.

The invention also relates to the use of a sensor device, as described in this document, for detecting an accumulation, in particular contamination, of volatile organic compounds (VOCs) in a gas atmosphere in an interior space of a laboratory apparatus for the treatment of liquid, in particular biological laboratory samples, in particular living cells, in particular in the gas atmosphere of the interior space of the incubator chamber of an incubator, the VOCs in each case preferably having been released by:
microbial organisms arranged in the interior space;
living cells arranged in the interior space;
Leakage from equipment components of the laboratory apparatus, in particular the incubator, especially the incubator chamber, into the interior;
   wherein the sensor device comprises at least one VOC sensor for detecting the VOCs and the at least one VOC sensor comprises at least one measurement area which is arranged in flow communication with the atmospheric gas of the interior. The at least one VOC sensor, its control and the evaluation of its measurement signals, can be carried out according to the description in this document, and can in particular be adapted to the requirements for the detection of the respective VOCs originating from the sources mentioned.

Further preferred embodiments of the incubator according to the invention, the sensor device and the method, result from the following description of the embodiment examples in connection with the figures and their description. Identical components of the embodiments are identified by substantially the same reference signs, unless otherwise described or otherwise apparent from the context. Showing:

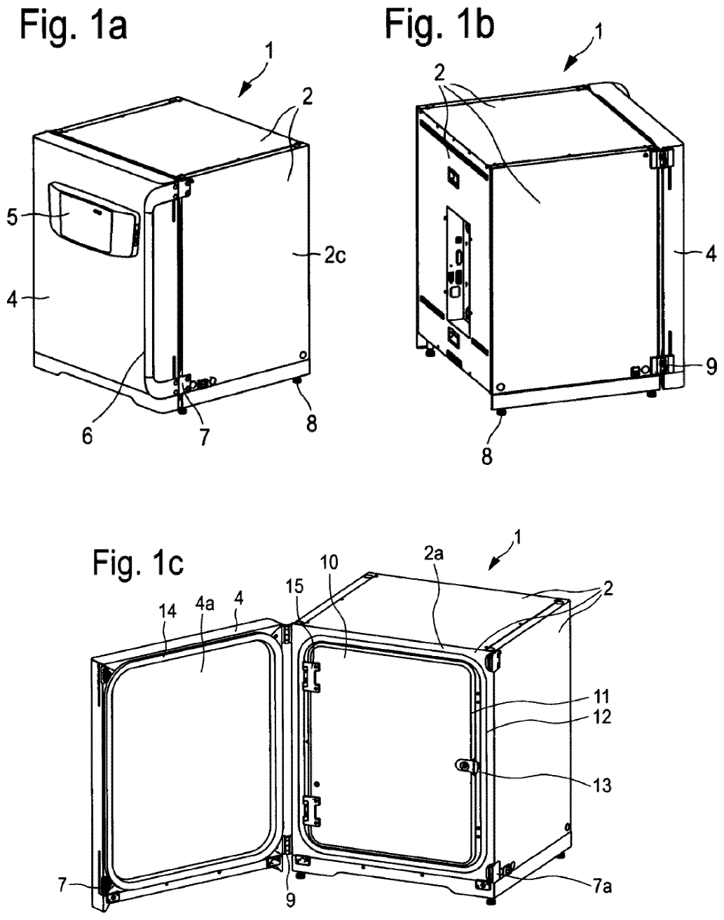

FIG. 1a shows a perspective side-front view of an incubator according to the embodiment of the invention, in the closed state of the housing door.

FIG. 1b shows a perspective side-rear view of the incubator of FIG. 1a.

FIG. 1c shows a perspective side-front view of the incubator of FIG. 1a, with the housing door open.

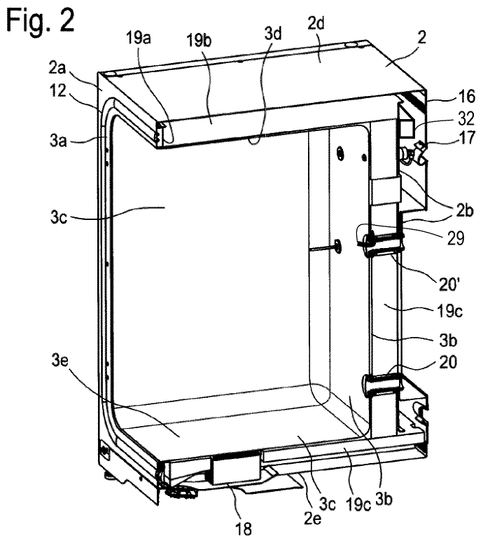

FIG. 2 shows a perspective side-front view of the incubator of FIG. 1a, with the housing door hidden and in a cross-section along a plane parallel to the side wall and centrally through the incubator.

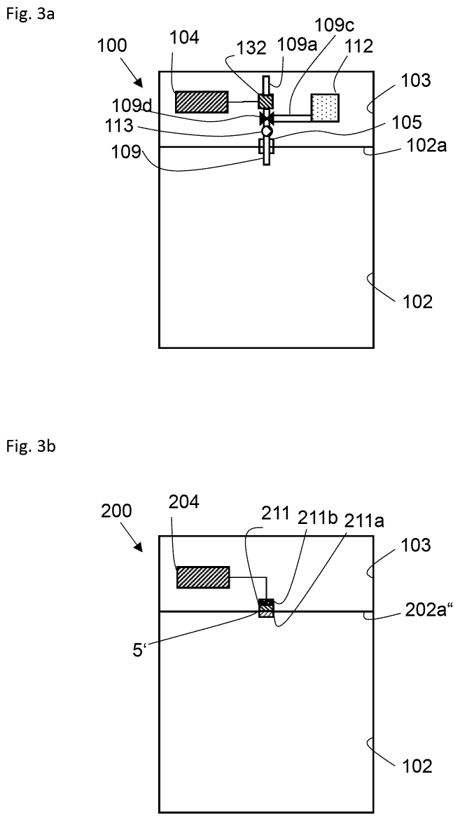

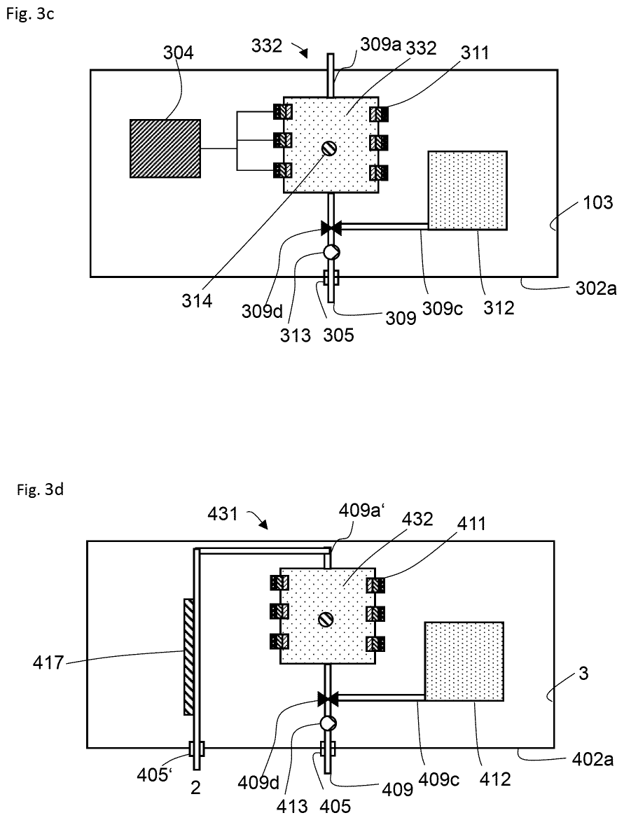

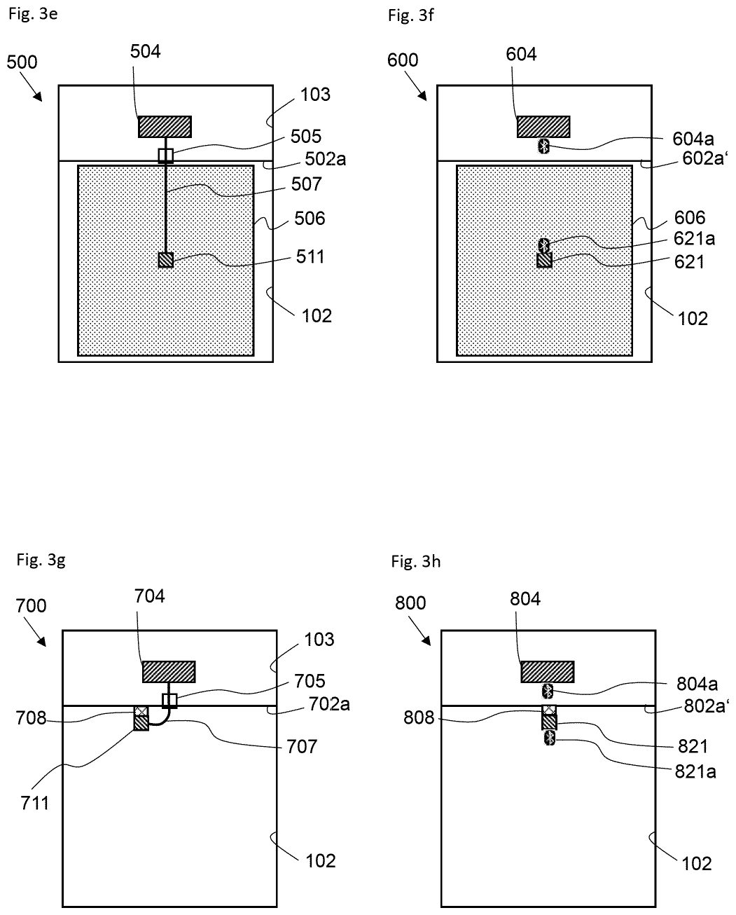

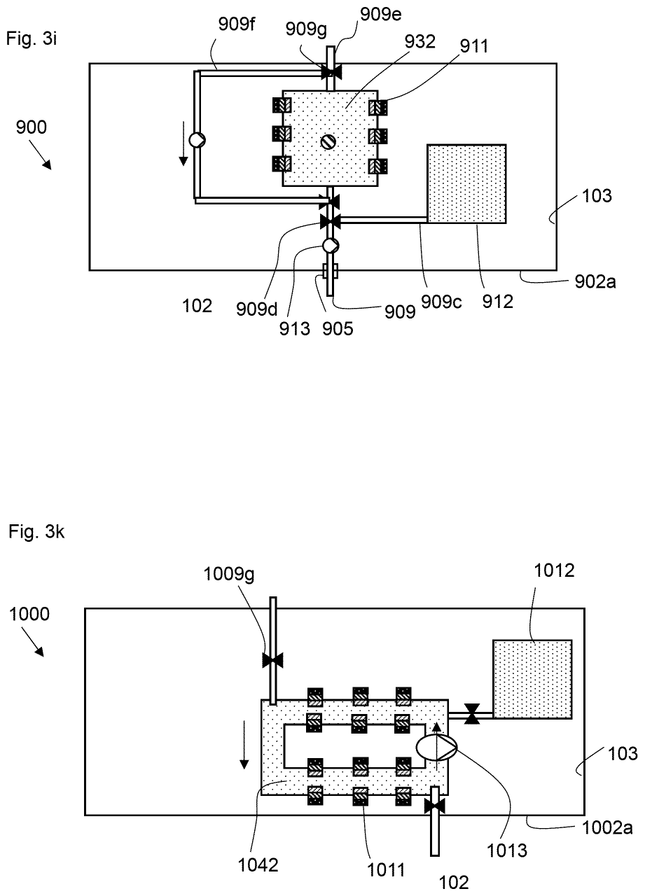

FIGS. 3a to 3k each show a different incubator according to a respective preferred embodiment of the invention.

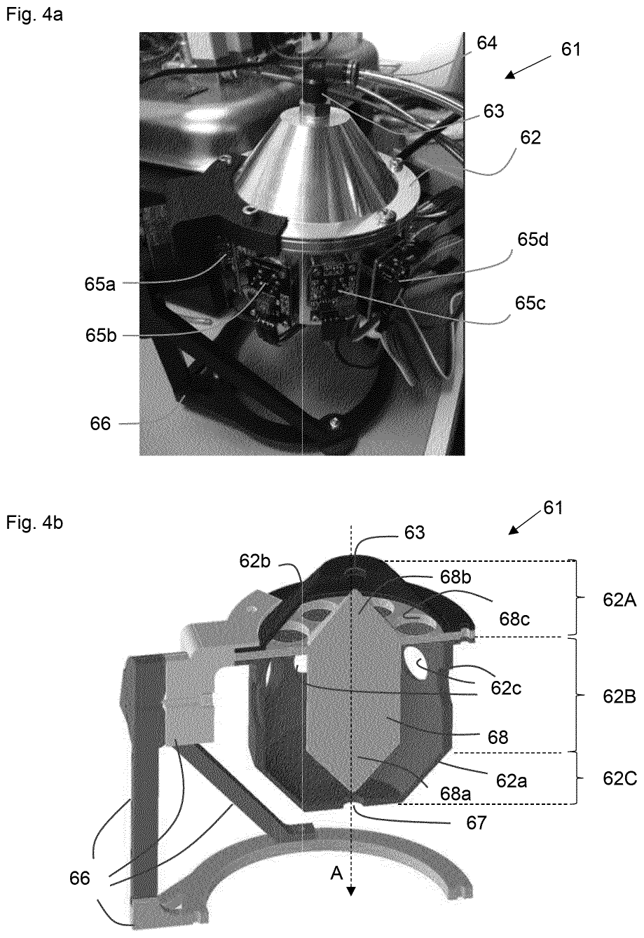

FIG. 4a shows a sensor device with measuring chamber according to an embodiment of the invention, which can be used in particular in the incubator 1 of FIGS. 1a to 2.

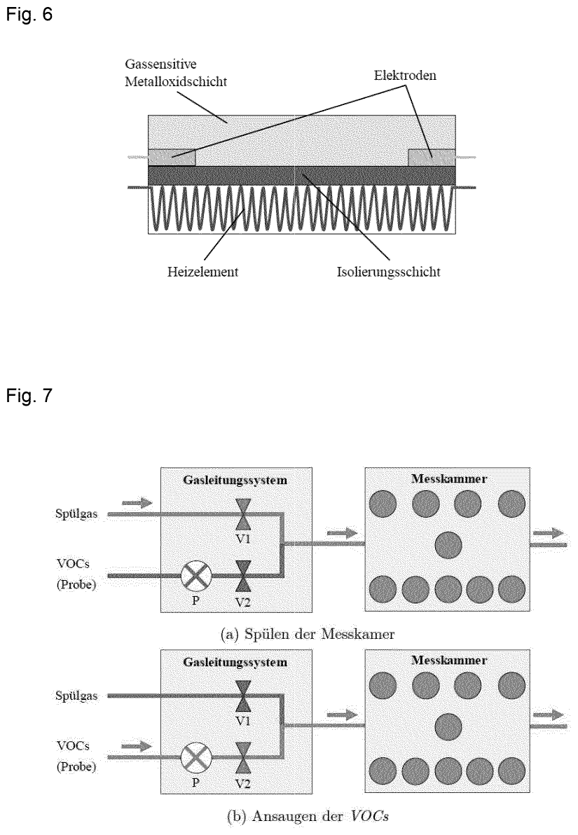

FIG. 4b shows the one sensor device with measuring chamber of FIG. 6a in a sectional view, without VOC sensors inserted and without their cables.

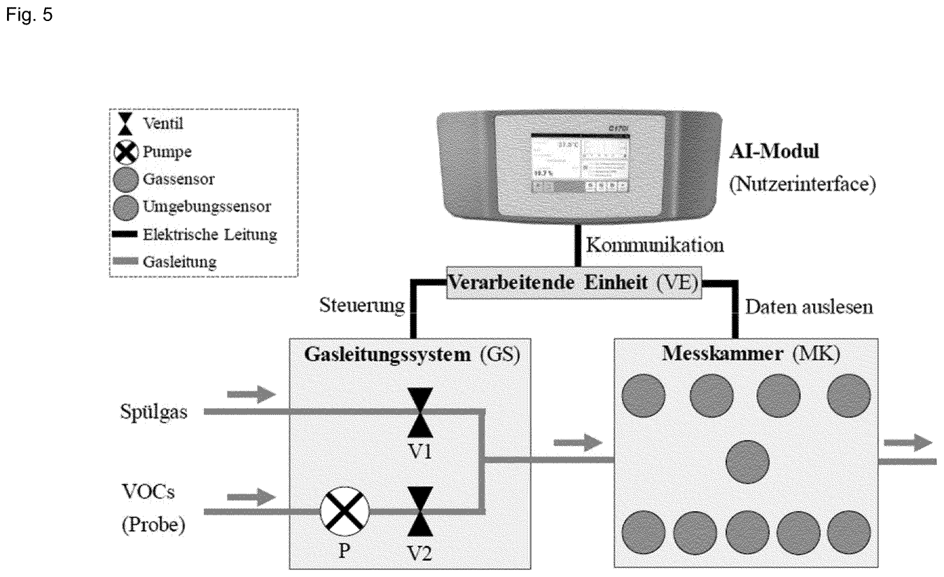

FIG. 5 shows: the schematic structure of the sensor device assignable to FIGS. 4a, 4b and its connection to a control device of the incubator.

FIG. 6 shows the schematic structure of a MOX sensor that can be used in a sensor device according to the invention.

FIG. 7 schematically shows the gas control for a VOC measurement that can be performed by the components shown in FIGS. 4a to 6.

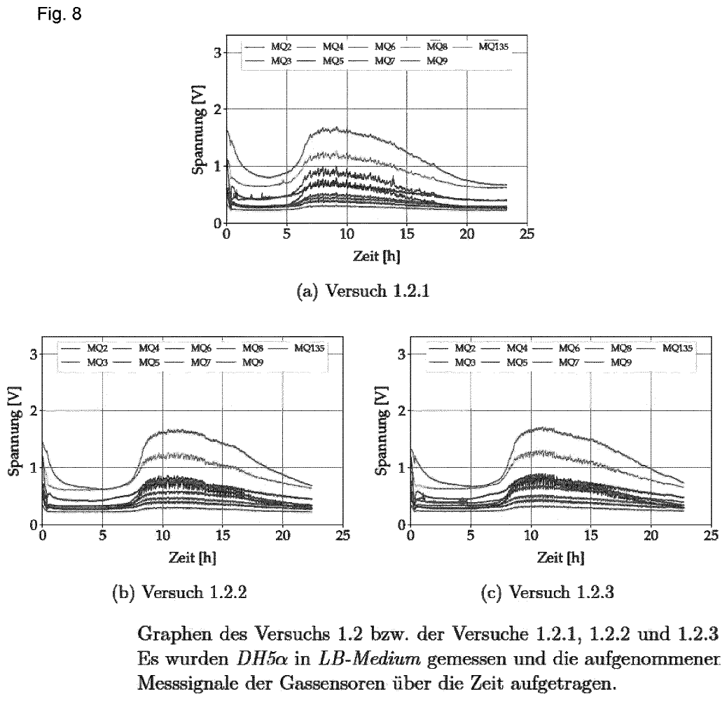

FIG. 8 shows diagrams with measured values for a measurement of VOCs from DH5□ grown in cell culture flasks in the incubator chamber of the incubator according to the invention.

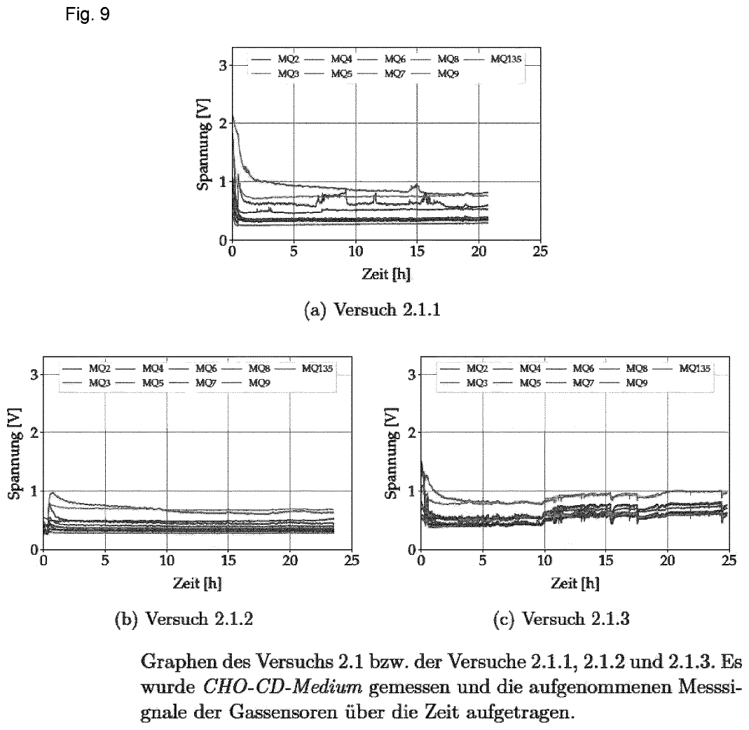

FIG. 9 shows diagrams with measured values for a measurement of VOCs from CHO-CD medium grown in cell culture flasks in the incubator chamber of the incubator according to the invention.

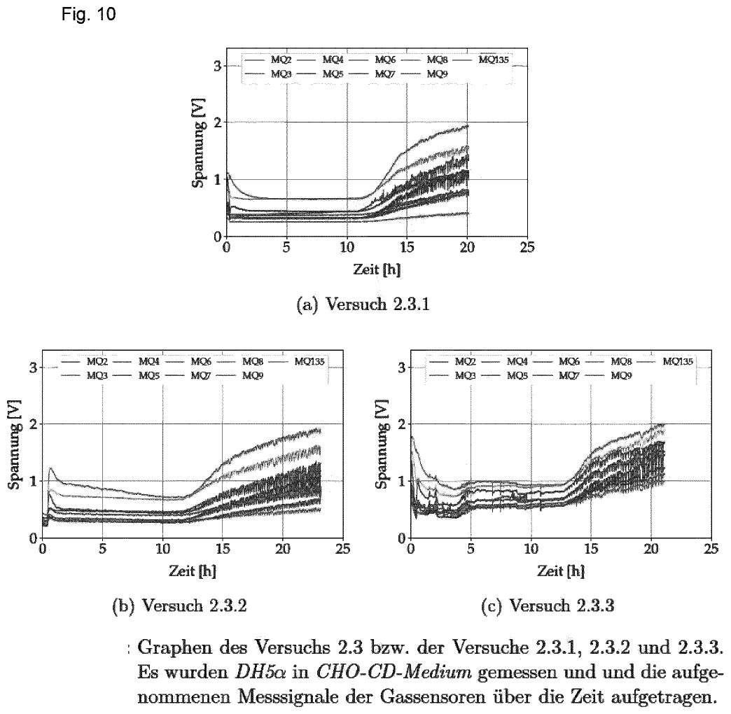

FIG. 10 shows graphs with measured values for a measurement of VOCs from DH5a in CHO-CD medium grown in cell culture flasks in the incubator chamber of the incubator according to the invention.

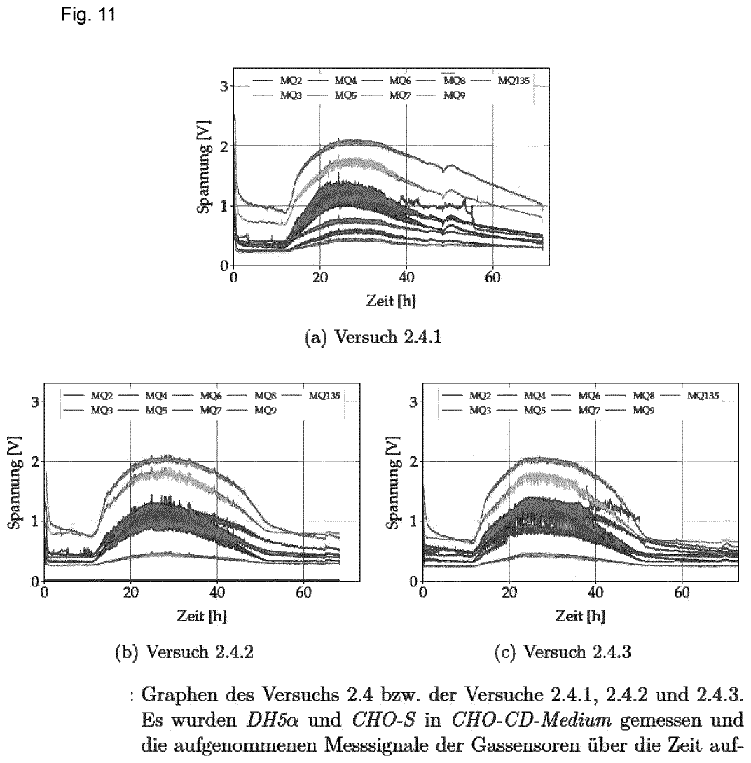

FIG. 11 shows plots of measured values for a measurement of VOCs from DH5□ and CHO-S in CHO-CD medium grown in cell culture flasks in the incubator chamber of the incubator of the invention.

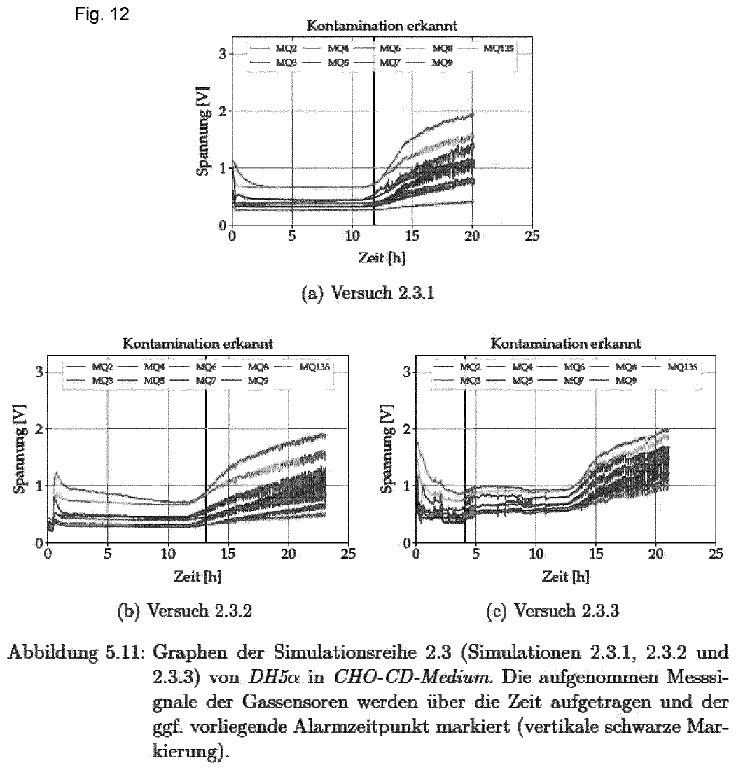

FIG. 12 shows diagrams with measured values for a measurement of VOCs from DH5a in CHO-CD medium, which grew in cell culture flasks in the incubator chamber of the incubator according to the invention. The recorded measurement signals of the gas sensors are plotted over time and the alarm point, if any, is marked (vertical black mark).

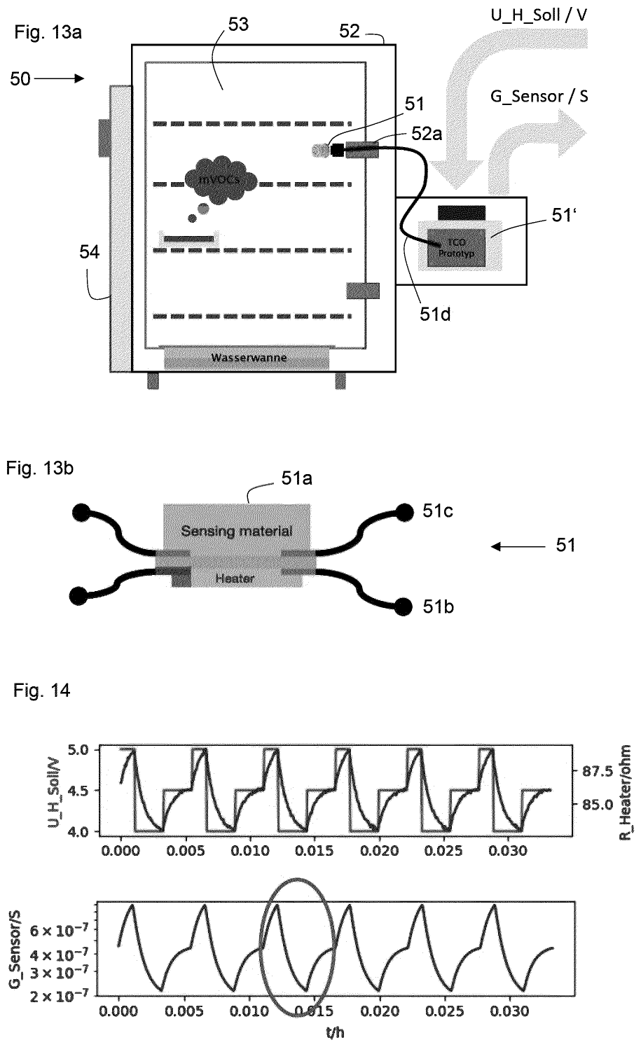

FIG. 13a shows an incubator according to the invention in accordance with a further preferred embodiment, with only one VOC sensor designed as a MOX sensor.

FIG. 13b schematically shows the MOX sensor of FIG. 13a.

FIG. 14 shows the stepwise progression of the periodic control (temperature cycled operation, TCO mode) of the MOX sensor of FIG. 13b and the resulting periodic progression of the electrical conductivity of the measuring range.

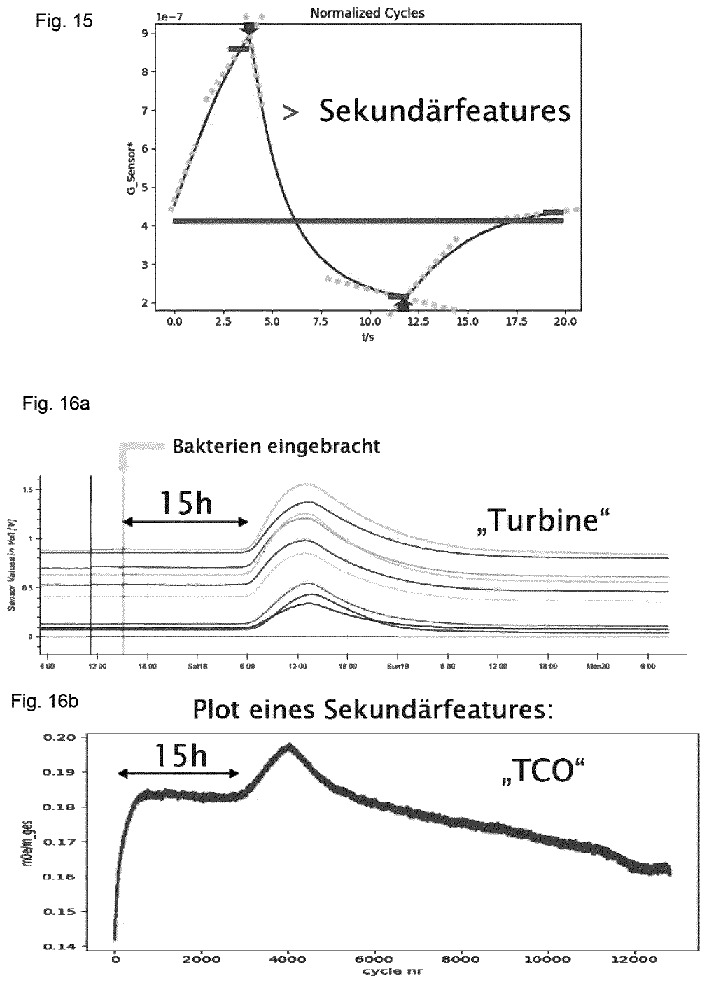

FIG. 15 shows the course of the measurement signals of a measurement period of a normalized measurement cycle, derived from a measurement signal curve in a measurement period in FIG. 14.

FIG. 16a shows the measurement signals from a sensor device designed as a turbine with several MOX sensors after a bacterial sample has been introduced into the interior of the chamber.

FIG. 16b shows the measurement signals from a single-sensor designed sensor device with a MOX sensor, according to FIG. 13a,b, after a bacterial sample was introduced into the chamber interior, as in FIG. 16a, where the measurement signal was determined in TCO mode and a secondary feature was used as the measurement signal.

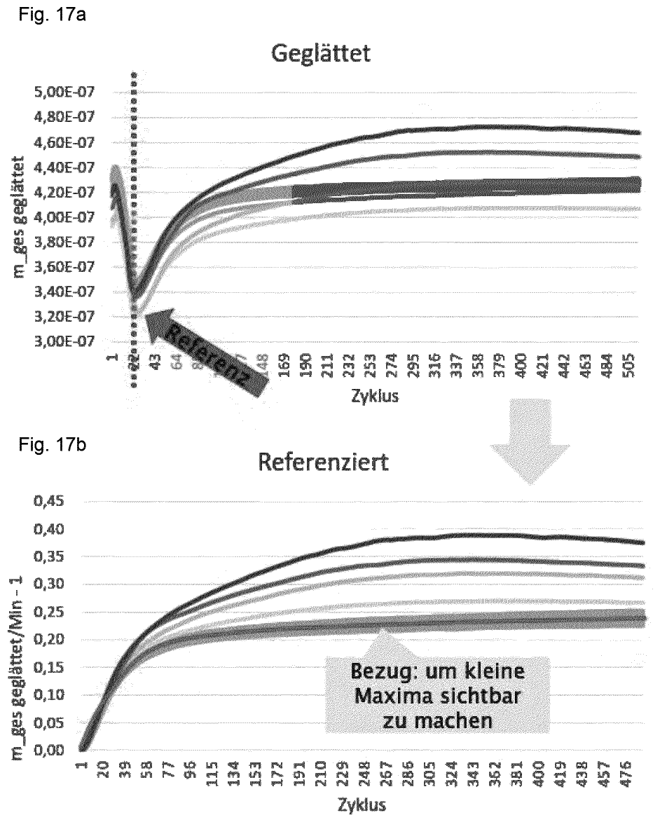

FIG. 17a shows the measurement signals of several experiments, from a sensor device designed with a single sensor with a MOX sensor, according to FIG. 13a,b, after an ethanol sample was introduced into the chamber interior, whereby a measurement point or a measurement signal was determined as the average value of the measurement signals of a measurement period in the TCO mode.

FIG. 17b shows the curves from FIG. 17b after a further evaluation involving mathematical transformations.

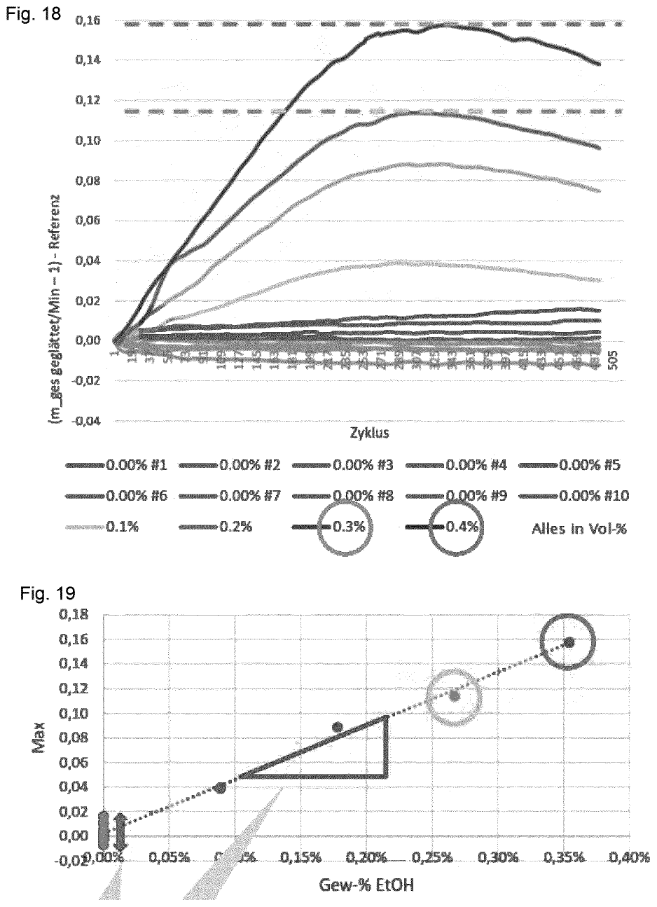

FIG. 18 shows the curves from FIG. 17b, after subtraction of a reference curve.

FIG. 19 shows the maxima of the curves from FIG. 18, plotted in a diagram whose abscissa shows the variable “alcohol concentration of the sample”.

FIG. 1a shows the incubator 1 for the growth of cell cultures, in this case a laboratory temperature control cabinet designed as a CO₂ incubator for the growth of eukaryotic cells. The incubator 1 comprises a housing 2 with a housing interior surrounded by at least one housing wall 2, and a temperature-controllable incubator chamber 3 (also “chamber 3”) arranged in the housing with a chamber interior surrounded by at least one chamber wall for receiving the laboratory samples. The outer walls of the housing are connected to each other in such a way that they support all other components of the incubator, in particular also the sensor device 30. The housing rests on pedestals 8. In the intended use, the outer sides of the side walls 2c of the housing, the front wall 2a, the rear wall 2b as well as the outer side of the housing door 4 and its inner side 4a, as well as the side walls of the chamber, the chamber front wall 3a, and the chamber rear wall 3b are arranged vertically, i.e. parallel to the direction of gravity. The upper outer side 2d and the non-visible bottom side of the housing and the bottom wall and top wall of the chamber are arranged horizon-tally accordingly. In the context of the description of the invention, the direction “downward” always refers to the direction of gravity with reference to which an incubator operated as intended is aligned; the direction “upward” is the opposite direction. The direction “towards the front” means the horizontal direction towards the front of the closed housing door, the direction “towards the back” means the horizontal direction towards the back of the incubator. The chamber is made of stainless steel, the housing is made of painted metal sheet.

The housing door 4 carries a user interface device 5, which here comprises a touch-sensitive display used by the user for reading and inputting information, in particular for outputting information obtained by means of the sensor device 20. The housing door comprises two hinges 9 which connect the housing door to the housing 2. By means of a locking device 7; 7a, 7b the housing door is held in the closed position. The housing door comprises a door handle 6.

In FIG. 1c, the chamber door 4 is shown open. The chamber door 10 is attached to the chamber front wall 3a by means of the hinges 15, and in the position shown is held closed by a hand latch 13 so that the chamber interior is not accessible. However, due to the transparency of the chamber door 10, the interior is visible to the user in this position. The chamber door is held gas-tight against the chamber front wall by a circumferential elastic seal 11 of the chamber door. The inside 4a of the housing door comprises a circumferential elastic seal 14 which, when the housing door is closed, lies flush against the housing front wall and the seal 12 circulating there and achieves gas-tight shielding of the area between chamber door 10 and housing door 4a.

As is partially visible in FIG. 2, the incubator has two temperature control devices which temper the chamber interior 3, i.e. set its temperature by means of temperature control. Some of the components 18 necessary for this purpose are arranged between the housing bottom wall 2e and the chamber bottom wall 3e. The heating coils of an upper heating circuit (not shown) are thermally coupled and connected to the outside of the chamber top wall 3d and an upper region of the chamber side walls, here approximately the upper 2/3 along the height of the side walls 3c of the chamber. The heating coils of a lower heating circuit (not shown) are thermally coupled and connected to the outside of the chamber bottom wall 3e and a lower region of the chamber side walls, here about the lower 1/3 along the height of the side walls 3c of the chamber.

A thermal insulation device 19 (19a, 19b, 19c) is provided between the chamber and the housing. It isolates the chamber, with temperature control equipment adjacent thereto, from the housing, which is in direct contact with the environment on its outside. The incubator normally operates at outside temperatures between 18° C. and 28° C. The temperature control devices or the temperature control system operate particularly efficiently in this range. The insulating device comprises a U-shaped curved insulating element 19b made of glass wool or mineral wool, which surrounds the chamber ceiling plate and the two chamber side walls 3c. It opens to the floor and to the rear wall at insulating panels 19c made of PIR foam (polyisocyanurate foam), and is terminated to the front side of the housing and chamber by a circumferential needlefelt strip 19a that abuts the inside of the housing front wall 2a, the chamber front wall 3a and the gasket 12. The thermal insulation of the chamber from the outside is optimized by the measures according to the invention.

A double housing rear panel 16 is attached to the housing rear panel 2b to cover rear-mounted components, in particular the measuring chamber 31 of a sensor device 30. The rear wall can be removed by means of a handle 17.

The incubator comprises two access ports 20, 20′ on its rear side, which allow lines, in particular at least one gas line between measuring chamber 32 and incubator chamber 3, and/or cables to be laid into the interior of chamber 3 through openings 20h, 20′h in the rear wall of the chamber, for example in order to control a sensor device optionally arranged in the interior. If an access port is not required, it is filled by a plug 25 made of thermally insulating material, e.g. silicone foam.

Preferably, a gas line 29 opens into the interior of the incubator chamber 3, passes through the opening 20h of the chamber rear wall and/or an opening in the port 20, between insulating material 19c of the thermal insulating device 19 along the chamber rear wall in order to be tempered by this indirectly tempered chamber wall, and only then passes away from the chamber rear wall, through the insulating material 19c into the preferably provided measuring chamber 32, which is arranged here in the area of the incubator 1 separated from the housing rear wall 16 and is connected to the latter. An exhaust air line of the measuring chamber 32 (not visible) preferably leads through the housing rear wall 16 into the surroundings of the incubator.

Preferred other embodiments of a sensor device, of at least one VOC sensor, and of a measuring chamber and their preferred arrangements on the incubator, in particular on the incubator 1, are described with reference to the following figures.

FIG. 3a shows: An incubator 200, with an incubator chamber 102 and a housing section 103 (“outer area”) of the incubator located outside the incubator chamber; each incubator in FIGS. 3a to 3k has these components. In FIG. 3a, by conveying the chamber atmosphere by means of conveying means 113 (pump or fan), gas exchange is effected from the inside of the chamber 102 by means of gas line 109 via port 105 through the chamber wall 102a to a measuring chamber 132 of the VOC sensor device 130, which is located with control 104 in the outside area 103, and exhaust gases of the VOC measuring chamber 132 are discharged to the environment via exhaust air line 109a; a purge gas 112 is supplied to the VOC measuring chamber via a purge gas line 109c and valve 109d to purge the VOC measuring chamber before a measurement.

FIG. 3b shows: an incubator 200: a VOC sensor control 204 is located in the outer area 103, the measurement area, namely the MOX side 211a of the VOC sensor 211 is located in the inner area of the chamber 102, a heating side 211b of the VOC sensor 211 is located in the outer area 103, the VOC sensor 211 is sealingly installed in hole 205′ in chamber wall 202a″.

FIG. 3c shows: an incubator 300: By pumping 313, gas is exchanged from the inside of the chamber 102 by means of gas line 309 via port 305 through the chamber wall 302a to a measuring chamber 332 of the VOC sensor device resp. e-nose 331 with its 6 VOC sensors 311, which is located with control 304 in the outer area 103, and exhaust gases from VOC measuring chamber 332 are discharged to the environment via exhaust air line 309a; purge gas 312 is conveyed to the VOC measuring chamber via purge gas line 309c and valve 309d before a measurement.

FIG. 3d shows: an incubator 400: By pumping 413, gas is exchanged from the inside of the chamber 102 by means of gas line 409 via port 405 through the chamber wall 402a to a measuring chamber 43 of the VOC sensor device or e-nose 431 with its 6 VOC sensors 411, which is located with control 404 in the outer area 103, and exhaust gases from the VOC measuring chamber 432 are conveyed via the exhaust air line 409a′ and optionally the filter 415 via the port 405′ back through the chamber wall 402a into the chamber 102; purge gas 412 is conveyed via the purge gas line 409c and the valve 409d to the VOC measuring chamber. Optionally, heating or heaters 417 of lines 409 are provided to prevent condensation.

FIG. 3e shows: an incubator 500: a VOC sensor 511 is located on a shelf 506 in the chamber 102 and connected by cable 507 and cable-based signal connection via port 505 through a chamber wall 502a to a VOC controller 504 in the exterior 103.

FIG. 3f shows: an incubator 600: a VOC sensor 621 is mounted on a shelf 606 in the chamber 10 and connected with wireless, radio 621a, 604a-based signal link through a port-free chamber wall 602a′ to a VOC controller 604 in the exterior 103.

FIG. 3g shows: an incubator 700: a VOC sensor 711 is attached to the chamber wall 702a inside the chamber 102 by means of magnetic staplers 708, and connected to a VOC controller 704 outside 103 by a cable 707-based signal connection via port 705 through the chamber wall 702a.

FIG. 3h shows: an incubator 800: a VOC sensor 821 is connected by magnetic stapler 808 in chamber 102 to chamber wall 802a′ with wireless, radio 821a, 804a-based signal connection through a port-free chamber wall 802a′ to a VOC controller 804 in exterior 103.

FIG. 3i shows: an incubator 900: shown is a measuring chamber with annular flow and circulating chamber gas: By pumping 913, the gas exchange takes place from the inside of the chamber 102 by means of the gas line 909 via the port 905 through the chamber wall 902 to a measuring chamber 932 of the VOC sensor device resp. e-nose 931 with its here 6 VOC sensors 911d, which is localized with the control 904 in the outer area 103, and after closing the valves 909d and 909g, exhaust gases from the VOC measuring chamber 932 are circulated via circulation lines 909f by means of pump 916 through the measuring chamber 932 to allow continuous convection of the VOC-containing chamber gas without major loss of chamber 102 gas resp. atmosphere; purge gas 912 is delivered to the VOC measurement chamber via a purge gas line 909c and valve 909d. As a result of the circulating volume of gas, no volume of gas is removed from chamber 102 beyond this volume, thereby minimizing chamber gas loss.

FIG. 3k shows: an incubator 1000: shown is a circularly designed measuring chamber for measurement on circulating gas: By means of pumps 1013 conveying, gas exchange takes place from the interior of the chamber 102 by means of the gas line 1009d via the port 1005 through the chamber wall 1002a to an annular measuring chamber 1042 of the VOC sensor device resp. e-nose 1041 with its here 12 VOC sensors 1011, and after closing the corresponding valves, exhaust gases are circulated through the VOC measuring chamber 1042 by means of the pump 1013 to allow continuous convection of the VOC-containing chamber gas without major loss of chamber 102 gas or atmosphere; a purge gas 1012 is conveyed to the VOC measuring chamber via the purge gas line 1009c and the valve 1009d. Advantages: the chamber gas is extracted from the chamber by the short-est route, so there is hardly any condensation; however, only the volume required for the narrow annular VOC measuring chamber has to be extracted from chamber 102; and during the measurement, the gas volume circulates; as a result, less chamber gas is lost compared to the setup with gas supply line and exhaust gas line to the atmosphere.

FIG. 4a shows a sensor device 61 with measuring chamber 62 according to an embodiment of the invention in a perspective side view from obliquely above, which can be used in particular in the incubator 1 of FIGS. 1a to 2. The measuring chamber is a hollow spindle-shaped body, which in FIG. 4a allows an inflow opening 63 at its upper end for the inflow of atmospheric gas from the incubator chamber by means of the gas line 64, and an outflow through the outflow line 67. The measuring chamber can be fastened to the incubator by means of a holder 66, in particular can be screwed, soldered or otherwise immovably connected to the incubator, in particular to a housing wall or chamber wall. The measuring chamber 62 has a partial hollow body 62a, into which a gas guide body 68 is placed, and which is covered by a cover part 62b. The parts 62a, 6sb and 68 are fixedly connected to each other. On the outside of the measuring chamber 62, one can see the backs 65a to 65i of the—here nine—MOX-VOC sensors, each of which has a measurement area or a heated MOX adsorption surface (not visible) on its front side, facing the central longitudinal axis A of the measuring chamber and arranged parallel to it.

FIG. 4b shows a view of the sensor device 61 with measuring chamber of FIG. 6a opened by sectioning the model in a perspective side view, without VOC sensors inserted and without their cables.

As shown in FIG. 4b, the measuring chamber 62 comprises an upper hollow cone-shaped section 62A, a middle hollow cylinder-shaped section 62B, and a lower hollow cone-shaped section 62C. At the top of the hollow cone-shaped section 62A, the inflow opening 63 for the gas is provided, and at the top of the hollow cone-shaped section 62C, the outflow opening 63 for the gas is provided. The main flow direction A results from the straight connection of the centers of the circular inflow opening 63 and the circular outflow opening 67. At the tip of the section 68b of the gas guide body facing the inflow opening, an environmental sensor (not visible; pressure, temperature, humidity) is arranged, which—as well as the nine VOC sensors, are connected to the electronic evaluation device for signal exchange.

The gas guide body is spindle-shaped and arranged coaxially to the axis A with the hollow spindle-shaped course of the outer wall of the measuring chamber 62 in such a way that a sleeve-shaped, or a flow channel with annular cross-sections results between the inside of the outer wall of the measuring chamber 62 and the outside of the gas guide body. In this way, the gas is guided uniformly and, in particular, with elimination of vortex formation—i.e. as laminarly as possible—past the measurement areas of the VOC sensors 65a-65i, which lie tightly against the circular openings 62c of the outer wall of the measuring chamber 62, so that at each of the nine openings 62c the same area of a MOX adsorption surface of the measurement area of the respective VOC sensor is in contact with the gas flowing past parallel to the direction of flow A. The gas is guided by the guide elements and the gas guide body. Due to the guide elements and the uniformity of the gas flow, the measurement performed by means of the sensor device 61 becomes particularly sensitive, and also reproducible and reliable. By arranging the rear sides of the MOX-VOC sensors outside the measuring chamber and not in contact with it, the heat transfer between the heating elements of the sensors and the measuring chamber is minimized, and the rear sides can also be easily cooled by convection/air flow.

### Incubator Embodiment Example with Sensor Device and Electronic Nose

The incubator according to the invention described below has the structure shown in FIGS. 1a to 2 and uses a sensor device 61 shown in FIGS. 4a, 4b (to implement the sensor device 30). The sensor device 61 is designed as an electronic nose with a total of nine different VOC sensors.

### Basic VOCs:

VOCs are released during the metabolism of microbial organisms and cells. The sensor device 61 is constructed according to the principle of an electronic nose and measures VOCs. It enables conclusions to be drawn about the contamination of a cell culture or about processes in cell cultures or in the incubator chamber that are associated with changes in the VOC concentration in the incubator chamber. The prerequisite for this is that the gas sensors are selective and sensitive enough for the VOCs that occur to provide evidence of microbial contamination.

A total of 9 gas sensors with different selectivities are installed here. These thus react differently to the VOCs of a microbial organism and thus generate a characteristic measurement signal pattern. The measurement takes place in particular during the period of biological sample growth, which is why the measurement signal pattern can be recorded as a function of time. The measurement signal pattern of the biological sample contains information that is to be analyzed and converted into a semantic statement. More details on the detectability of microbial contamination of a cell culture will be described below. Each of these nine gas sensors can also be used in a sensor setup with fewer than nine gas sensors or with only a single gas sensor. In addition, not only VOCs from a microbial organism can be detected, but also VOCs resulting from release of device components from the incubator immediately after manufacture.

The variety of gas sensors allows a better differentiation between different microbial organisms and cells. For example, in some cases a similar measurement signal pattern is produced for different contaminants, but the signal characteristics of individual gas sensors differ characteristically. The advantage of using several different gas sensors is therefore the increase in the information content of the measurement.

The sensor device 61 has a measuring chamber (MK), a gas conduction system (GS) and a processing unit (VE). The MK contains the gas sensors (VOC sensors) which measure the VOCs. The GS directs the VOCs to the MK using actuators. The VE controls the GS, reads the gas sensors and the ambient sensor, processes the data, and provides a communication interface to the incubator. The VE includes an electronic control device of the sensor device, which includes the evaluation device comprising a data processing device. Here, a microcontroller of the control device is the Raspberry Pi 3 B (Raspberry). The communication interface enables information flow between an AI module and measurement chamber, and thus control of the sensor device 61 using the user interface.

FIG. 5 shows: the schematic structure of the sensor device 61 and its connection to a control device of the incubator, also referred to as the AI module. The MK includes the gas sensors, the ambient sensor, an input and an output. The GS directs either purge gas or VOCs (i.e., chamber atmosphere gas) into the MK with the aid of gas lines from the valves and pump. The VE is connected to the GS and the MK via electrical lines. It controls the GS, reads out the gas sensors and the ambient sensor, processes the data, and provides a communication interface to the incubator. The communication interface enables the flow of information between the AI module and the sensor device 61 and thus the control of the sensor device 61 with the aid of the user interface (user interface device).

To ensure that the same starting conditions are created as far as possible before each VOC analysis in this embodiment, the MK is purged before the VOCs are introduced. The purging process is advantageous for generating comparable measurement results. If the sensor device 61 is not used, either gases from the environment can enter the MK or VOCs from the past VOC measurement can remain in the MK. Preferably, a purge gas of constant composition is always used. To keep the proportion of changing gases in the purge gas low, nitrogen 5.0 is preferably used as the purge gas. This has a purity level of >99.999%.

The MK comprises an inlet and outlet (inflow and outflow). Flushing gas or VOCs are fed into the MK via the inlet. A Y-coupling is located upstream of the inlet, which combines the purge and VOC lines. The gases escape from the MK again via the outlet. The inlet and outlet are preferably located opposite and centrally on a respective outer wall of the MK in order to ensure the most uniform gas flow and distribution possible.

MOX sensors are preferably used as gas sensors (VOC sensors).

Various analytical methods exist to detect VOCs, including the electronic nose. An Electronic Nose uses a sensor array to generate a fingerprint for a given odor using pattern recognition and distinguish it from fingerprints of other odors. In this way, an electronic nose mimics the olfactory system of mammals and allows odors to be recognized as a whole and the source of the odor to be identified. For example, microorganisms can be identified by drawing conclusions about the source based on the detected mixture of characteristic VOCs.

The measuring system of an electronic nose is in particular built up from a sample con-ducting unit, detection unit as well as calculation unit and the used gas sensors are preferably selected in such a way that these are sensitive for the occurring gas molecules, but the individual gas sensors react differently strongly to these. Here, metal oxide semiconductor (MOX) gas sensors are used, which belong to the class of chemical sensors. Chemical sensors comprise a detection layer, with the help of a chemical interaction can be transformed into an electrical signal and are also not only inexpensive, but can also be used in continuous measurement operation.

The design and operation of MOX sensors: The sensor mechanism is based on the fact that, depending on the concentration of the target gas, the electrical conductivity of the gas-sensitive metal oxide layer or semiconductor is changed and thus the presence as well as the quantity of the target gas is determined. Typically, a MOX sensor consists of four elements: Gas sensitive metal oxide layer, electrodes, heating element and insulating layer (see FIG. 6).

FIG. 6 shows the schematic structure of a MOX sensor. A MOX sensor consists of 4 elements: Gas sensitive metal oxide layer, contact electrodes, heating element and insulation layer. The heating element is separated from the gas-sensitive metal oxide layer and the contact electrodes by the insulating layer. The gas-sensitive metal oxide layer is heated by the heating element and oxygen molecules from the environment are adsorbed on the surface of the gas-sensitive metal oxide layer. The adsorbed oxygen molecules capture electrons from the conductive bands of the semiconductor and energetic barriers are formed, thus blocking part of the electron flow in the semiconductor and thus degrading the electrical conductivity or increasing the resistance of the gas sensor. As soon as reducing gases (target gases) are present, they react with the bound oxygen molecules. The oxygen molecules are released from the surface of the gas-sensitive metal oxide layer and the conductivity increases or the resistance decreases.

Manufacturers usually specify ambient conditions in which the MOX sensors provide valid measured values and how much they are influenced by them. In order to have an overview of how much the environmental conditions have an influence on the VOC measurements, an environmental sensor is also placed in the center of the MK. The relevant environmental conditions include humidity, temperature and ambient pressure.

The process of a VOC measurement is preferably divided into two phases—the rinsing and the introduction of the VOCs. The incubator with sensor equipment is first initialized for the measurement, flushing is performed. The gas sensors are preferably continuously read and temporarily stored for the measurement. If required, the user can permanently save the temporarily stored data and export it if necessary. The VOC measurement itself is started or stopped by the user or automatically by the incubator by controlling the GS.

The elements to be controlled are the valves and the pump. To start the VOC measurement, the flushing process is initiated in the example. Valve 1 (V1) is open, valve 2 (V2) is closed and the pump (P) is deactivated (see FIG. 7 above). When the flushing process is to be terminated and the supply of VOCs is to be started, valve 1 (V1) is closed, valve 2 (V2) is opened and the pump (P) is started (see FIG. 7 below). To stop the VOC measurement, the pump (P) is switched off and valve 2 (V2) is closed. The sensor device is preferably supplied with voltage via a power supply unit of the incubator. This applies to the VE, the gas sensors of the MK, the valves and the pump of the GS.

FIG. 7 schematically shows the gas control during a VOC measurement. This is preferably divided into flushing and aspiration of VOCs. Gas conveying components are marked in green and non-conveying components in red. During the purging process, valve 1 is open, valve 2 is closed, the pump is deactivated and the purging gas flows through the measurement chamber. During the suction of VOCs, valve 1 is closed, valve 2 is open, the pump is activated and the VOCs flow through the measuring chamber.

The MK consists of an aluminum injection-molded chamber with a screw-on cover and contains the gas sensors of different types (MQ 1, MQ 2, MQ 3, MQ 4, MQ 5, MQ 6, MQ7, MQ 8, MQ9 and MQ135 or reference marks 65a-i; conventionally obtained from HANWEI ELETRONICS CO., LTD) and the environmental sensor (BME680). The ambient sensor provides the required environmental parameters of temperature, humidity, and pressure and was placed inside the MK. The gas sensors were chosen to be mostly selective for the potentially occurring groups of substances of VOCs and were placed adjacent to the MK according to the established concept. The connection points between gas sensors and MK were sealed with silicone. The respective selectivity of the gas sensors can be seen in Table 1 under the Details column.

TABLE 1
Hardware components of the measurement chaber (MC)
Measurement chamber (MC)

| Component | Designation | Details |
|---|---|---|
| Gas sensor | MQ 2 | Alkans (Butan, Propan, Methan), |
| | | Alcohols, Hydrogen |
| Gas sensor | MQ 3 | Alcohols |
| Gas sensor | MQ 4 | Alkans (Methan) |
| Gas sensor | MQ 5 | Alkans (Butan, Propan, Methan) |
| Gas sensor | MQ 6 | Alkans (Butan, Propan, Methan) |
| Gas sensor | MQ 7 | Oxids (Carbon monoxid) |
| Gas sensor | MQ 8 | Hydrogen |
| Gas sensor | MQ 9 | Carbon monoxid, Alkans (Butan, Propan, Methan) |
| | | Alcohols, Benzenes (Benzene), Amins (Ammonia), |
| Gas sensor | MQ 135 | Oxids (Carbon- and Nitrogendioxid) |
| Environmental sensor | Adafruit BME680 | Temperature, Humbidity, Pressure |
| Injection-molded chamber | Hammond | Aluminum, 170 × 120 × 55 mm |
| Silicon | | Sealing gas sensors |

Escherichia coli bacteria of strain ΔH5α (ΔH5α) were used as a test sample to demonstrate the functionality of the sensor device and to generate contamination in the incubator chamber. These are commonly encountered in everyday laboratory work. Various VOCs, are emitted by the ΔH5α, see Table 2. The VOCs belong to the substance groups of benzenes, alkylbenzenes, ketones, alcohols, alkanes, terpenes, acids, carboxylic acids, esters, aldehydes, alkenes, heterocyclic amines and indoles. The largest proportion of the listed VOCs belongs to the alcohol group of substances. According to the manufacturer's specifications, some of the gas sensors used are selective for gases belonging to the substance groups of alcohols, alkanes, benzenes and amines. Accordingly, the gas sensors MQ2, MQ3, MQ4, MQ5, MQ6, MQ9 and MQ135 should respond to the VOCs of DH5 and an increase in the measurement signals should be noted. Since the largest proportion of the VOCs produced belong to the alcohol group, the gas sensors MQ2, MQ3 and MQ135 generate higher measurement signals than the other gas sensors.

TABLE 2
DH5α emitting VOCs (in UAPC-Notation) and their
respective chemical classification [4]

| IUPAC-Name | Chemical Classification |
|---|---|
| 1,2,3-Trimethylbenzene | Benzene, Alkylbenzenes |
| 1-(4-Methylphenyl)ethanone | Benzene, Ketones |
| 1,4-Xylene | Benzene, Alkylbenzenes |
| 2-(4-Methyl-3-cyclohexene-1-yl)-2-propanol | Alcohols, Terpenes |
| 2-Ethyl-1-hexanol | Alcohols |
| 2-Phenylethanol | Alcohols |
| 3-Hydroxy-2-butanon | Alcohols, Ketones |
| 2,3-Butandiol | Alcohols |
| 2-Decanol | Alcohols |
| Dodecan | Alkanes |
| Octadecan | Alkanes |
| Hexanacid | Acids, Carbonacids |
| Nonaacid | Acids, Carbonacids |
| Octanacid | Acids, Carbonacids |
| Ethyl-octanoat | Ester |
| 3-Methylbutyl-acetat | Ester |
| Lauraldehyd | Aldehydes |
| 2-Methylpentanal | Aldehydes |
| 4-Methyl-1-hexen | Alkenes |
| 1H-Indol | Indoles, hetrerocyclic Amines |

Chinese Hamster Ovary cell cultures (CHO) are cell types frequently encountered in everyday laboratory work. The VOCs emitted by CHO belong to the substance groups of alkanes, aldehydes, esters, benzenes, ketones, pyrazoles, oximes and alcohols. The largest of the listed VOCs belongs to the substance group of alkanes. According to the manufacturer's data, some of the gas sensors used are selective for gases belonging to the substance groups of alcohols, alkanes and benzenes. Accordingly, the gas sensors MQ2, MQ3, MQ4, MQ5, MQ6, MQ9, and MQ135 should respond to the VOCs of the CHOs and show an increase in the measurement signals. Since the majority of the VOCs produced belong to the alkane group, the gas sensors MQ2, MQ4, MQ5, MQ6, and MQ9 should produce higher measurement signals than the other gas sensors. As with the DH5, the cell cultures must exhibit consistent growth dynamics in each experiment. Unlike the DH5, the CHOs were not grown independently, but were provided by the applicant and grown according to internal standard procedures.

The evaluation device (VE) uses an algorithm to detect whether or not contamination is present in a test sample (CHO with/without ΔH5α). The algorithm developed is based on the sequential CUSUM analysis technique (also called CUSUM Control Chart) and was first presented by Page. An AI algorithm, particularly a neural network would also be possible for evaluation. The CUSUM analysis technique is used to monitor the deviations of a running process. xᵢ be the i-th observation of the process. The process is classified into two states—either it is under control or not. When the process is under control, xᵢ is subject to a normal distribution with a mean μ0 and a standard deviation σ. μ0 is often interpreted as the target value that xᵢ must be as close to as possible for the process to remain under control.

FIG. 8 shows plots of measured values for a measurement of VOCs from ΔH5α grown in cell culture flasks in the incubator chamber of the incubator according to the invention. ΔH5α in LB medium was measured and the recorded measurement signals from the gas sensors were plotted against time. To record the measurement data shown in FIG. 8 (analogously: FIGS. 9, 10), a control device of the incubator, in particular by means of programming a data processing device of the incubator, carries out the following steps: within a first period from the start of the measurement at time zero, the measurement chamber, which has the measurement ranges of the MOX sensors of different types, is purged with a purge gas, in this case nitrogen. During this first period, the MOX sensors (each in the form of a voltage value) measure reference measured values essentially in parallel and successively, while the purging gas flows past the measurement areas. This first period is 5 hours in FIGS. 8a and 7 hours in FIGS. 8b and 8c. In a second period immediately following the first period, a gas line serving as a supply air duct is opened by means of a valve, which simultaneously stops the inflow of purge gas into the measuring chamber. As a result, a volume of the gas atmosphere flows into the measuring chamber—and out of it again, for example, through an exhaust air duct. The volume flows past the measurement areas during the second period. Within this second time period, the MOX sensors measure measured values essentially in parallel and successively. The second time period lies in FIG. 8a between the end of the 5th hour, in FIGS. 8b and 8c between the end of the 7th hour from the start of the measurement to the end of the period 22.5 hours from the start of the measurement in each case. For each MOX sensor, the reference measured values determined within the first time period form a “baseline” in comparison to which the measured value is considered: the difference between the measured value and the reference measured value at a certain point in time can be considered as the result of the measurement of the contamination (result measurement data). If this difference is zero, there is no contamination. If it is greater than zero here in the example, contamination is present. Most MOX sensors detect contamination by non-zero result measurement data.

In this type of measurement (FIG. 8, 9, 10) and subsequent evaluation, the data storage device is programmed to perform the following steps: (i) storing a measurement data set in a data memory, which here includes measurement values of the number N=9>1 of VOC sensors, a measurement value being characteristic of the detected measurement signal of the respective VOC sensor, which was detected in the presence of a volume of the gas atmosphere originating from the incubator chamber and present at the measurement area of the VOC sensor; ii) determining first result measurement data from a comparison of the measurement data set with a reference data set, in particular using a difference of the measurement data set and the reference data set, which contains reference measurement values, in particular recorded as a function of time, of the number N>1 of VOC sensors, a reference measurement value being characteristic of the detected measurement signal of the respective VOC sensor, which was recorded in the presence of a purge gas originating from a purge device and present at the measurement range of the VOC sensor;

Optionally, the step of: iii) recognizing a characteristic data pattern in the result measurement data set containing the result measurement data may also be provided, wherein the characteristic data pattern represents a specific VOC or VOC mixture detected in the atmospheric gas, in particular also its concentration or quantity. For example, the result measurement data of the MOX sensors of different types, in particular taking into account a common scaling factor, or taking into account a normalization factor, can record the characteristic data pattern at one time or at several times of the measurement.

FIG. 9 shows plots of measured values for a measurement of VOCs from CHO-CD medium grown in cell culture flasks in the incubator chamber of the incubator according to the invention. CHO-CD medium were measured using the sensor device 61 and the recorded measurement signals from the gas sensors were plotted against time.

FIG. 10 shows plots of measured values for a measurement of VOCs from DH5a in CHO-CD medium grown in cell culture flasks in the incubator chamber of the incubator according to the invention. DH5a in CHO-CD medium was measured and the recorded measurement signals from the gas sensors were plotted against time.

FIG. 11 shows plots of measured values for a measurement of VOCs from DH5a and CHO-S in CHO-CD medium grown in cell culture flasks in the incubator chamber of the incubator according to the invention. DH5a in and CHO-S in CHO-CD medium were measured and the recorded measurement signals from the gas sensors were plotted against time.

FIG. 12 shows diagrams with measured values for a measurement of VOCs from DH5a in CHO-CD medium, which grew in cell culture flasks in the incubator chamber of the incubator according to the invention. The recorded measurement signals of the gas sensors are plotted over time and the alarm time, if any, is marked (vertical black marking).

Based on the experimental results, it is shown that sensor device 61 is capable of detecting a growing microbial contamination of a cell culture. Thus, the gas sensing system can help ensure that microbial contaminants are not remain undetected and thus further problems in the application areas of cell cultivation are avoided. The integration of the gas sensing system into the CO₂ incubator was successful and the use of the gas sensing system in the laboratory environment of the CO₂ incubator was facilitated.

### Example of an Incubator with a Sensor Device Comprising Only One VOC Sensor

FIG. 13 shows an incubator 50 for incubating living cell cultures, comprising a housing 52, therein an incubator chamber 53 for receiving objects, in particular cell culture containers, in an interior space of the incubator chamber which can be closed by means of the incubator door 54 and which has a controllable gas atmosphere, a sensor device for detecting an accumulation, in particular a contamination caused, of volatile organic compounds (VOCs) in the gas atmosphere of the interior space, the sensor device comprising precisely one VOC sensor 51 for detecting the VOCs and the VOC sensor 51 comprising a measuring range 51a. The VOC sensor 51 is a thick film sensor, MOX sensor, commercially available under the name Figaro TGS2602 through Figaro USA, Inc. Such a sensor can also be used in an incubator with a sensing device comprising more than one VOC sensor. This also applies to the aspects of its mounting in the incubator, the control by means of constant or variable voltage and the evaluation of the measurement signals.

The sensor 51 is fixedly mounted on the inner wall of the chamber 53 so that the metal oxide surface serving as the measurement area is in flow communication with the atmospheric gas of the inner chamber. An electric cable 51d leads through a port 52a of the chamber wall into a housing area of the housing 52, in which an electric control device 51′ is arranged, to which the sensor 51 is connected by means of the cable 51d. By means of the control device 51′, the sensor 51 is controlled and evaluated. The heating element of the sensor 51 is operated by the control device with a measuring voltage U_H_Soll, measured in volts, which are applied to electrodes 51b of the sensor 51 (FIG. 13b). An electrical resistance value, for example an electrical resistance R_Sensor, measured in ohms, or an electrical conductivity G_Sensor, measured in siemens, is also detected via electrodes 51c. The corresponding measurement signal is evaluated by the control device 51′. It is possible that the value of the electrical resistance output by the sensor is output as a voltage in volts, wherein the desired measured value R_Sensor or G_Sensor can be derived from this output value in accordance with a previously known dependency, in particular a proportionality; in particular, this output voltage value is proportional to the desired measured value R_Sensor or G_Sensor.

The control device 51′ serves as an evaluation device, and includes the data processing device. It is programmable with a program code, and programmed to perform the following steps, in particular according to this program code:
Receiving a measurement signal R/G_Sensor, in particular measurement data, of the MOX sensor; in particular: Receiving a sequence of measurement signals in time, in particular for the duration of a measurement time Δτ, one after the other, which in particular form the time course of the measurement of the MOX sensor;
Comparing of the measured signal with a reference value;
Decide, based on the result of this comparison, whether there is a change in the VOC concentration in the gas atmosphere of the interior, especially a change characteristic of contamination of the interior.

The control device 51′ is arranged to control the heating element of the sensor 51 with a periodically changing voltage U-H-Soll in order to generate a corresponding periodically changing temperature at the metal oxide surface. This mode of operation of a sensor device is also referred to as “temperature cycled operation” (“TCO”). For this purpose, the heater is controlled here with a voltage U-H-Soll comprising a step-like progression, which provides several different values per heating period T, here the voltage values 4.0 volts, 4.5 volts and 5.0 volts. This is shown in FIG. 14. Each of these voltage values is set for a predetermined portion of the heating period T. The heating period is preferably between 5 seconds [s] and 30 s, preferably between 15 s and 25 s, preferably between 17 s and 23 s, and here is 20 s. The control results in a periodically changing measurement signal R/G_Sensor with the measurement period T.

In the case of a periodic measurement signal, the evaluation is preferably carried out by statistically evaluating one or preferably several periods of the measurement signal. Preferably, the data processing device is programmed to determine an average course of a measurement period. This may involve superposing the values of a number M of measurement periods and then multiplying this added period course by the inverse number 1/M. In this way, a measurement signal is smoothed and the influence of measurement artifacts is reduced.

Preferably, the data processing means is programmed to derive from the signal of a single measurement period or from an average course of a measurement period at least one secondary value relating to a characteristic of the measurement period referred to as a secondary feature. As shown in FIG. 15, a secondary value can be a slope that is present at a characteristic time of the measurement period, for example, the time of the changeover of the voltage value. In FIG. 15, it can be seen that the characteristic slope can be recorded just before or after these times. For the purpose of further evaluation, the secondary value can be compared with at least one reference value for this secondary value, as described.

Preferably, the data processing device is programmed to determine an average value of several measurement signals, in particular, to determine an average value of several or essentially all measurement signals of a measurement period. For the purpose of further evaluation, the mean value can be compared with at least one reference value for this mean value, as described.

FIG. 16a shows the time course of measured values determined using the sensors of a sensor device 61 of the “turbine” type. The measurement signal of each sensor reacts to the addition of the bacteria, i.e. the voc source, to the chamber interior with a time delay, in that an increase in the measurement signal can be detected after approx. 15 hours. In FIG. 16b, a single sensor (Figaro, as shown in FIGS. 13a, 13b) was operated in TCO mode in the comparable experiment. A measuring point is in each case an average value of a cycle or a measuring period T=20 s, which was specified according to a sensor control as shown in FIG. 14. A secondary feature of the measuring signal period was evaluated, in this case the quotient mOe/m_ges. The letter sequence “mOe” describes a secondary feature. Each measuring cycle consists of several temperature cycles. Each temperature cycle has a number starting with 0. Each temperature cycle has a start “s” and an end “e”. There are two classes of features: mean “m” and slope “s”. Accordingly, the quotient means: m0e=mean value at the end of the zero cycle/m_ges=mean value over a whole measuring cycle.

FIG. 17a shows, superimposed for better comparison, a series of different curves of evaluated measurement signals (electrical conductivity of the measurement layer, in Siemens) acquired in different experiments. The measurement setup corresponds to the device of FIGS. 13a, b, which was operated in TCO mode, measurement period 20 s. The curves distinguishable by their color or graduation correspond to one experiment each. The volume percentage of ethanol (e.g., between 0.1% and 0.4%) was varied in an ethanol-water mixture of predetermined total volume, which was always placed in the same open container inside the chamber. The reference time is the moment when the chamber door was closed again after the sample containers were briefly placed inside. In FIG. 17a, for each cycle (measuring period) the—here smoothed—average value “m_ges smoothed” of the measuring signals of the measuring period is plotted. In FIG. 17b, the value “m_ges smoothed” is additionally divided by the minimum value of the respective curve at the reference time, and the value 1 is subtracted from this quotient (m_ges smoothed/Min−1) to normalize the measurement signals. All measurement signals are normalized to this value (mges/min) —in this case the minimum is then equal to 1. If 1 is then subtracted from the measurement values, the curve is drawn to zero.

In FIG. 18, the maxima of the measurement curves from FIG. 17b are plotted against the respective alcohol concentration (ethanol in water, wt %). On the one hand, it can be seen that the measurement arrangement is efficiently suited to detect and quantify the EtOH-VOC in the chamber interior by means of the chemical MOX sensor, because the maxima of the evaluated quantity (m_ges smoothed/Min−1) lie on a straight line. From any other comparable experiment (m_ges smoothed/Min−1) of unknown ethanol concentration, the ethanol concentration of the sample can be determined from the position of the maximum of the curve and the slope of the straight line. The experiment is highly relevant to the detection of bacteria in a chamber interior, since a major component of the VOC's released during bacterial metabolic processes are alcohols. The straight line slope itself is also a measure of the sensitivity of the measurement setup—larger slopes imply easier discrimination of the evaluated variable. An optional estimation of a detection limit, the detection limit and the determination limit for the measuring arrangement/measurement method can be determined by means of DIN 32645. With regard to FIG. 18, the slope is taken into account, the drift of the measurement signals over time, and the noise of the measurement signals.

1. An incubator for incubating live cell cultures, comprising
an incubator chamber for receiving objects, in particular cell culture containers, in a closable interior of the incubator chamber, which comprises a controllable gas atmosphere,
a sensor device for detecting an accumulation, in particular contamination, of volatile organic compounds (VOCs) in the gas atmosphere of the interior, the sensor device comprising at least one VOC sensor for detecting the VOCs and the at least one VOC sensor comprising at least one measurement area which is arranged in flow communication with the atmospheric gas of the interior.

2. The incubator according to claim 1, comprising a flow channel, in particular at least one gas line, which leads from the interior of the incubator chamber into a measuring chamber arranged in an exterior space of the incubator chamber, so that atmospheric gas is transportable from the incubator chamber into the measuring chamber.

3. The incubator according to claim 2, comprising a flow channel, in particular an exhaust duct, arranged to convey an exhaust air from the measuring chamber to an outer space of the measuring chamber.

4. The incubator according to claim 2, comprising a flow channel, in particular a return gas line, arranged to convey the exhaust air from the measuring chamber, preferably through a filter, in particular a hepa filter, back into the incubator chamber.

5. The incubator according to claim 2, wherein the sensor device comprises a plurality of VOC sensors whose measurement areas, in particular whose adsorption areas, are arranged in contact with an interior of the measuring chamber.

6. The incubator according to claim 1, wherein the at least one of the VOC sensors are MOX sensors and comprise a heating side, which in particular are each arranged outside the measuring chamber.

7. The incubator of claim 2, wherein the measurement chamber comprises a toroidal interior of a toroidal measurement chamber portion of the measurement chamber, wherein the measurement areas of a plurality of VOC sensors are arranged along a wall of the toroidal measurement chamber portion.

8. The incubator according to claim 2, wherein the sensor device is configured as an electronic nose and comprises an electronic control device and a plurality of different VOC sensors, and in particular comprises a flushing device by means of which the measuring chamber can be flushed by a flushing gas.

9. The incubator of claim 8, wherein the control device comprises a data processing device comprising at least one data memory programmed to perform the following steps:
i) storing a measurement data record in a data memory which contains the measured values, in particular recorded as a function of time, of the number N>1 of VOC sensors, a measured value being characteristic of the detected measurement signal of the respective VOC sensor which was recorded in the presence of a volume of the gas atmosphere originating from the incubator chamber and applied to the measurement range of the VOC sensor;
ii) determining first result measurement data from a comparison of the measurement data set with a reference data set, in particular using a difference of the measurement data set and the reference data set, which includes, in particular time-dependently detected, reference measurement values of the number N>1 of VOC sensors, a reference measured value being characteristic of the detected measurement signal of the respective VOC sensor, which was recorded in the presence of a purge gas originating from a purge device and applied to the measurement range of the VOC sensor;
optionally:
iii) recognizing a characteristic data pattern in the result measurement data set containing the result measurement data, wherein the characteristic data pattern represents a specific VOC detected in the atmospheric gas, in particular also its concentration or amount.

10. The incubator according to claim 9, wherein step iii) includes using a classification algorithm determined by machine learning, in particular a neural network, to classify the characteristic data pattern.

11. The incubator of claim 8, wherein the control device comprises a data processing device comprising at least one data memory programmed to perform the following steps:
i) storing a test measurement data record in a data memory which contains test measurement values, in particular recorded as a function of time, of the number N>1 of VOC sensors, a test measurement value being characteristic of the detected measurement signal of the respective VOC sensor which was recorded in the presence of a volume, supplied to the measurement chamber and applied to the measurement range of the VOC sensor, of a previously known test gas comprising a VOC content which is previously known, in particular in terms of type and/or quantity;
ii) determining second result measurement data from a comparison of the test measurement data set with a reference data set, in particular using a difference of the test measurement data set and the reference data set, which contains the reference measurement values, in particular recorded as a function of time, of the number N>1 of VOC sensors, a reference measured value being characteristic of the detected measurement signal of the respective VOC sensor, which was recorded in the presence of a purge gas taken from a purge device and applied to the measurement range of the VOC sensor;
iii) storing a second result measurement data set containing the second result measurement data and comprising a now known data pattern characteristic of the test gas.

12. The incubator of claim 11, wherein a step iv) is provided that includes using the second result measurement data as labeled data to train an adaptive classification algorithm by machine learning, in particular a neural network, that is subsequently usable for classifying measured characteristic data patterns.

13. The incubator according to claim 1, comprising an information output system, in particular a display, a loudspeaker or a data interface to an external data-processing device, for outputting information about this detection in dependence on the detection of VOCs detected by means of the sensor device, in particular for outputting warning information to a user or a monitoring system.

14. A laboratory monitoring system for detecting the accumulation of VOCs, in particular for detecting contamination, in an incubator chamber, comprising
at least one incubator according to claim 1;
at least one data-processing device arranged externally to the at least one incubator, which is in particular in a data exchange connection with the at least one incubator, in particular via an intranet or the internet;
wherein the data-processing device is programmed to acquire the measurement data about a possible contamination of the incubator chamber obtained from the at least one incubator and determined by means of the sensor device of the incubator by the detection and to store said measurement data in a data storage device, in particular in order to communicate said measurement data to a further device, in particular to a mobile radio device.

15. A method for detecting the accumulation of VOCs, in particular for detecting the contamination, in the interior of an incubator chamber of an incubator, in particular an incubator according to claim 1, comprising the steps:
acquisitioning of measurement data by means of a sensor device of the incubator, which comprises at least one VOC sensor for detecting volatile organic compounds (VOCs), the VOC sensor comprising a measurement area which is arranged in flow communication with the atmospheric gas of the interior;
determining of possible contamination of the gas atmosphere of the interior by evaluation of the measurement data.

16. A retrofittable sensor device for detecting a possible accumulation of VOCs, in particular contamination, in the gas atmosphere of the interior of an incubator chamber, wherein the sensor device comprises at least one VOC sensor for detecting volatile organic compounds (VOCs), wherein the VOC sensor comprises a measuring range which is arrangeable in flow communication with the atmospheric gas of the interior space, and wherein the sensor device preferably comprises a gas line arrangeable between the interior space and the measurement area, and preferably a pump for conveying a volume of the gas atmosphere of the interior space of the incubator chamber through the gas line to the measurement area.

17. An incubator arrangement, comprising an incubator with an incubator chamber and a sensor device according to claim 16, which is arranged for detecting a possible accumulation of VOCs, in particular a contamination, in the gas atmosphere of the interior of an incubator chamber, on the incubator or in the incubator chamber of the incubator.
